(19) United States
(12) Patent Application Publication
(10) Pub. No.: US 2024/0261436 A1
(43) Pub. Date: Aug. 8, 2024

(54) GENE EDITING THERAPY FOR HIV INFECTION VIA DUAL TARGETING OF HIV GENOME AND CCR5

(71) Applicant: TEMPLE UNIVERSITY - OF THE COMMONWEALTH SYSTEM OF HIGHER EDUCATION, Philadelphia, PA (US)

(72) Inventors: Kamel KHALILI, Bala Cynwyd, PA (US); Rafal KAMINSKI, Philadelphia, PA (US)

(21) Appl. No.: 18/566,468

(22) PCT Filed: Jun. 2, 2022
(86) PCT No.: PCT/US22/31941

§ 371 Date: Dec. 1, 2023

Related U.S. Application Data
(60) Provisional application No. 63/196,045, filed on Jun. 2, 2021.

(51) Int. Cl. A61K 48/00; A61K 31/4985; A61K 31/505; A61K 31/52; A61K 31/7068; A61P 31/18; C12N 9/22; C12N 15/113

(52) U.S. Cl. CPC A61K 48/005; A61K 31/4985; A61K 31/505; A61K 31/52; A61K 31/7068; A61P 31/18; C12N 9/22; C12N 15/1132; C12N 15/1138; C12N 2310/20

(57) ABSTRACT

Compositions for specifically cleaving target sequences in retroviruses include nucleic acids encoding a Clustered Regularly Interspace Short Palindromic Repeat (CRISPR) associated endonuclease and a guide RNA sequence complementary to a target sequence in a retrovirus and a receptor used by a retrovirus for infecting a cell. The CRISPR construct edits, for example, proviral HIV DNA, thereby eliminating the provirus from an infected cell and simultaneously edits a viral receptor, e.g. CCR5 preventing infection and reinfection of the host.

### CROSS REFERENCE TO RELATED APPLICATIONS

This application claims the benefit of U.S. Provisional Application No. 63/196,045, filed Jun. 2, 2021, and U.S. Provisional Application No. 61/049,683, filed May 1, 2008, which is incorporated herein by reference in its entirety.

### STATEMENT REGARDING FEDERALLY SPONSORED RESEARCH

This disclosure was made with U.S. government support under a grant awarded by the National Institutes of Health (NIH) to Kamel Khalili (R01MH110360). The U.S. government may have certain rights in the disclosure.

### FIELD OF THE DISCLOSURE

The present disclosure relates to compositions and methods that target a retroviral genome and a viral receptor, for example human immunodeficiency virus (HIV). The compositions, which can include nucleic acids encoding a Clustered Regularly Interspace Short Palindromic Repeat (CRISPR) associated endonuclease and a guide RNA sequence complementary to a target sequence in a human immunodeficiency virus and/or a viral receptor can be administered to a subject having or at risk for contracting an HIV infection.

### BACKGROUND

For more than three decades since the discovery of HIV-1, AIDS remains a major public health problem affecting greater than 35.3 million people worldwide. AIDS remains incurable due to the permanent integration of HIV-1 into the host genome. Current therapy (highly active antiretroviral therapy or HAART) for controlling HIV-1 infection and impeding AIDS development profoundly reduces viral replication in cells that support HIV-1 infection and reduces plasma viremia to a minimal level. But HAART fails to suppress low level viral genome expression and replication in tissues and fails to target the latently-infected cells, for example, resting memory T cells, brain macrophages, microglia, and astrocytes, gut-associated lymphoid cells, that serve as a reservoir for HIV-1. Persistent HIV-1 infection is also linked to co-morbidities including heart and renal diseases, osteopenia, and neurological disorders. There is a continuing need for curative therapeutic strategies that target persistent viral reservoirs.

Current therapy for controlling HIV-1 infection and preventing AIDS progression has dramatically decreased viral replication in cells susceptible to HIV-1 infection, but it does not eliminate the low level of viral replication in latently infected cells which contain integrated copies of HIV-1 proviral DNA. There is an urgent need for the development of for curative therapeutic strategies that target persistent viral reservoirs, including strategies for eradicating proviral DNA from the host cell genome.

### SUMMARY

The present disclosure provides compositions and methods relating to treatment and prevention of retroviral infections, for example, the human immunodeficiency virus HIV-1. The compositions and methods target the retroviral genome, a viral receptor or combinations thereof.

Specifically, the present disclosure provides compositions including a nucleic acid sequence encoding a CRISPR-associated endonuclease, and one or more isolated nucleic acid sequences encoding gRNAs, wherein each gRNA is complementary to a target sequence in a retroviral genome. In a preferred embodiment, two or more gRNAs are included in the composition, with each gRNA directing a Cas endonuclease to a different target site in integrated retroviral DNA. In some embodiments, at least one endonuclease targets a viral receptor, such as for example, CCR5 receptors. In another embodiment, a composition comprises two of more endonucleases targeted to a retroviral genome and two or more endonucleases targeted to a virus receptor.

In some embodiments, an expression vector comprises an isolated nucleic acid sequence encoding a CRISPR-associated endonuclease, and one or more isolated nucleic acid sequences encoding gRNAs, wherein each gRNA is complementary to a target sequence in a retroviral genome and/or a receptor used by a virus to attach to and/or infect a cell.

In certain embodiments, at least one antiretroviral agent is administered to the subject prior to administering the at least one gene editing agent. In certain embodiments, the at least one antiretroviral agent and at least one gene-editing agent are co-administered. In certain embodiments, the at least one antiretroviral agent and at least one gene-editing agent are administered sequentially.

In certain embodiments, the CRISPR-associated endonuclease is a Type I, Type II, or Type III Cas endonuclease. In certain embodiments, the CRISPR-associated endonuclease is a Cas9 endonuclease, a Cas12 endonuclease, a Cas 13 endonuclease, a CasX endonuclease, a CasΦ endonuclease or variants thereof. In certain embodiments, the CRISPR-associated endonuclease is a Cas9 nuclease or variants thereof. In certain embodiments, the Cas9 nuclease is a Staphylococcus aureus Cas9 nuclease. In certain embodiments, the Cas9 variant comprises one or more point mutations, relative to wildtype Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In certain embodiments, a Cas9 variant comprises a human-optimized Cas9; a nickase mutant Cas9; saCas9; enhanced-fidelity SaCas9 (efSaCas9); SpCas9(K855a); SpCas9(K810A/K1003A/r1060A); SpCas9(K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A. In certain embodiments, the CRISPR-associated endonuclease is optimized for expression in a human cell.

In certain embodiments, the isolated nucleic acid sequences are included in at least one expression vector selected from the group consisting of: a lentiviral vector, an adenovirus vector, an adeno-associated virus vector, a vesicular stomatitis virus (VSV) vector, a pox virus vector, and a retroviral vector. In certain embodiments, the expression vector comprises: a lentiviral vector, an adenoviral vector, or an adeno-associated virus vector. In certain embodiments, the adeno-associated virus (AAV) vector is AV1, AAV2, AAV3, AAV4, AAV5, AAV6, AAV7, AAV8, AAV9, AAV10, AAV11, AAVDJ, or AAVDJ/8. In certain embodiments, the vector comprising the nucleic acid further comprises a promoter. In certain embodiments, the promoter comprises a ubiquitous promoter, a tissue-specific promoter, an inducible promoter or a constitutive promoter.

In certain embodiments, a composition for preventing or treating a retroviral infection in vitro or in vivo, the composition comprises at least two isolated nucleic acid sequences wherein: the first isolated nucleic acid sequences encodes a first Clustered Regularly Interspaced Short Palindromic Repeat (CRISPR)-associated endonuclease and at least one guide RNA (gRNA), the gRNA being complementary to a target sequence in the integrated retroviral DNA; the second isolated nucleic acid sequences encodes a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell in vitro or in vivo; wherein the CRISPR-associated endonuclease, the first gRNA, and the second gRNA are capable of excising intervening sequences between the first target sequence and the second target sequence. In certain embodiments, the first isolated nucleic acid sequences encode at least one gRNA, the gRNA being complementary to a target sequence in the integrated retroviral DNA and a second gRNA that is complementary to a second target sequence in the integrated retroviral DNA. In certain embodiments, the second isolated nucleic acid sequence encodes a first gRNA that is complementary to a first target sequence in a gene encoding for at least one receptor used by a retrovirus for attachment and/or infection of a cell; and a second gRNA that is complementary to a second target sequence in a gene encoding for at least one receptor used by a retrovirus for attachment and/or infection of a cell. In certain embodiments, the first isolated nucleic acid sequence encodes a first gRNA, the gRNA being complementary to a target sequence in the integrated retroviral DNA and a second gRNA that is complementary to a target sequence in a gene encoding for at least one receptor used by a retrovirus for attachment and/or infection of a cell. In certain embodiments, the at least one receptor comprises CCR5, variants or combinations thereof.

In certain embodiments, the composition further comprises one or more isolated nucleic acid sequence encoding one or more (CRISPR)-associated endonucleases and at least one guide RNA (gRNA) having complementarity to one or more target sequences, the target sequences comprising retroviral DNA sequences. In certain embodiments, the target sequences comprise one or more nucleic acid sequences in HIV comprising: long terminal repeat (LTR) nucleic acid sequences, Gag nucleic acid sequences, nucleic acid sequences encoding structural proteins, non-structural proteins or combinations thereof.

In certain embodiments, the gRNAs comprise a nucleic acid sequence having at least a 75% sequence identity to gRNA sequences: CCR5-A: CGGCAGCATAGTGAGCCCAG (SEQ ID NO: 1), CCR5-B: TCAGTTTACACCCGATCCAC (SEQ ID NO: 2); LTR1: GCAGAACTACACACCAGGGCC (SEQ ID NO: 3), GagD: GGATAGATGTAAAAGACACCA (SEQ ID NO: 4) and combinations thereof. In certain embodiments, the gRNAs comprise CCR5-A: CGGCAGCATAGTGAGCCCAG (SEQ ID NO: 1), CCR5-B: TCAGTTTACACCCGATCCAC (SEQ ID NO: 2); LTR1: GCAGAACTACACACCAGGGCC (SEQ ID NO: 3), GagD: GGATAGATGTAAAAGACACCA (SEQ ID NO: 4) and combinations thereof.

In certain embodiments, the composition further comprises a therapeutically effective amount of at least one antiretroviral agent. In certain embodiments, the antiretroviral agent is formulated as a long-acting slow effective release (LASER) antiretroviral agent. In certain embodiments, the at least one antiretroviral agent is nanoformulated. In certain embodiments, the at least one antiretroviral agent comprises: myristolyated dolutegravir, lamivudine, abacavir, rilpivirine or combinations thereof.

In certain embodiments, a composition comprises at least two isolated nucleic acid sequences wherein: the first isolated nucleic acid sequence encodes a first Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell; the second isolated nucleic acid sequence encodes a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell; wherein the CRISPR-associated endonuclease, the first gRNA, and the second gRNA are capable of excising intervening sequences between the first target sequence and the second target sequence. In certain embodiments, the at least one receptor used by a retrovirus for attachment and/or infection of a cell comprises a chemokine receptor. In certain embodiments, the at least one chemokine receptor comprises CCR5, variants or combinations thereof. In certain embodiments, the composition further comprises two or more isolated nucleic acid sequence encoding one or more (CRISPR)-associated endonucleases and at least two guide RNAs (gRNAs) having complementarity to one or more target sequences, the target sequences comprising retroviral DNA sequences, wherein the CRISPR-associated endonuclease and gRNAs are capable of excising intervening sequences between the first target sequence and the second target sequence. In certain embodiments, the target sequences comprise one or more nucleic acid sequences in HIV comprising: long terminal repeat (LTR) nucleic acid sequences, Gag nucleic acid sequences, nucleic acid sequences encoding structural proteins, non-structural proteins or combinations thereof. In certain embodiments, further comprising a therapeutically effective amount of at least one antiretroviral agent. In certain embodiments, the antiretroviral agent is formulated as a long-acting slow effective release (LASER) antiretroviral agent. In certain embodiments, the at least one antiretroviral agent is nanoformulated. In certain embodiments, at least one antiretroviral agent comprises: myristolyated dolutegravir, lamivudine, abacavir, rilpivirine or combinations thereof.

In certain embodiments, a method of inactivating or eradicating an integrated retroviral DNA and preventing infection by a retrovirus in vitro or in vivo, includes the steps of exposing the cell to a composition comprising at least one isolated nucleic acid sequence encoding a gene editing complex comprising a Clustered Regularly Interspaced Short Palindromic Repeat (CRISPR)-associated endonuclease, a first guide RNA (gRNA), the first gRNA being complementary to a target sequence in the integrated retroviral DNA; a second guide RNA (gRNA), the second gRNA being complementary to a target sequence in a gene encoding for at least one receptor used by a retrovirus for attachment and/or infection of a cell in vitro or in vivo, wherein the CRISPR-associated endonuclease, the first gRNA, and the second gRNA are capable of excising intervening sequences between the first target sequence and the second target sequence. In certain embodiments, the method further comprises administering to a subject composition comprising a therapeutically effective amount of at least one antiretroviral agent. In certain embodiments, the antiretroviral agent is formulated as a long-acting slow effective release (LASER) antiretroviral agent. In certain embodiments, the at least one antiretroviral agent is nanoformulated. In certain embodiments, the at least one antiretroviral agent comprises: myristolyated dolutegravir, lamivudine, abacavir, rilpivirine or combinations thereof. In certain embodiments, the at least one antiretroviral agent is administered to the subject prior to administering the at least one gene editing agent. In certain embodiments, the at least one antiretroviral agent and at least one gene-editing agent are co-administered. In certain embodiments, the at least one antiretroviral agent and at least one gene-editing agent are administered sequentially. In certain embodiments, the at least one receptor used by a retrovirus for attachment and/or infection of a cell comprises a chemokine receptor. In certain embodiments, the at least one chemokine receptor comprises CCR5, variants or combinations thereof. In certain embodiments, the method further comprises administering two or more isolated nucleic acid sequence encoding one or more (CRISPR)-associated endonucleases and at least two guide RNAs (gRNAs) having complementarity to one or more target sequences, the target sequences comprising retroviral DNA sequences, wherein the CRISPR-associated endonuclease and gRNAs are capable of excising intervening sequences between the first target sequence and the second target sequence. In certain embodiments, the target sequences comprise one or more nucleic acid sequences in HIV comprising: long terminal repeat (LTR) nucleic acid sequences, Gag nucleic acid sequences, nucleic acid sequences encoding structural proteins, non-structural proteins or combinations thereof.

In certain embodiments, a method of inactivating or eradicating an integrated retroviral DNA and preventing infection by a retrovirus in vitro or in vivo, comprising administering at least two isolated nucleic acid sequences wherein: the first isolated nucleic acid sequence encodes a first Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell; the second isolated nucleic acid sequence encodes a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell; wherein the CRISPR-associated endonuclease, the first gRNA, and the second gRNA are capable of excising intervening sequences between the first target sequence and the second target sequence. In certain embodiments, the at least one receptor used by a retrovirus for attachment and/or infection of a cell comprises a chemokine receptor. In certain embodiments, the at least one chemokine receptor comprises CCR5, variants or combinations thereof. In certain embodiments, the method further comprises administering one or more isolated nucleic acid sequence encoding one or more (CRISPR)-associated endonucleases and at least one guide RNA (gRNA) having complementarity to one or more target sequences, the target sequences comprising retroviral DNA sequences. In certain embodiments, the target sequences comprise one or more nucleic acid sequences in HIV comprising: long terminal repeat (LTR) nucleic acid sequences, Gag nucleic acid sequences, nucleic acid sequences encoding structural proteins, non-structural proteins or combinations thereof. In certain embodiments, the method further comprises administering a therapeutically effective amount of at least one antiretroviral agent. In certain embodiments, the antiretroviral agent is formulated as a long-acting slow effective release (LASER) antiretroviral agent. In certain embodiments, the at least one antiretroviral agent is nanoformulated. In certain embodiments, the at least one antiretroviral agent comprises: myristolyated dolutegravir, lamivudine, abacavir, rilpivirine or combinations thereof. In certain embodiments, the at least one antiretroviral agent is administered to the subject prior to administering the at least one gene editing agent. In certain embodiments, the at least one antiretroviral agent and at least one gene-editing agent are co-administered. In certain embodiments, the at least one antiretroviral agent and at least one gene-editing agent are administered sequentially. In certain embodiments, the at least one receptor used by a retrovirus for attachment and/or infection of a cell comprises a chemokine receptor, CD4. In certain embodiments, the at least one chemokine receptor comprises CCR5, CXCR4, variants or combinations thereof.

In certain embodiments, the sequences encoding structural proteins comprise nucleic acid sequences encoding: Gag, Gag-Pol precursor, Pro (protease), Reverse Transcriptase (RT), integrase (In), Env or combinations thereof, and wherein the sequences encoding non-structural proteins comprise nucleic acid sequences encoding: regulatory proteins, accessory proteins or combinations thereof. In certain embodiments, regulatory proteins comprise: Tat, Rev or combinations thereof, and wherein accessory proteins comprise Nef, Vpr, Vpu, Vif or combinations thereof.

Any compositions or methods provided herein can be combined with one or more of any of the other compositions and methods provided herein.

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, the preferred materials and methods are described herein. In describing and claiming the present disclosure, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof. The term also refers to non-nucleoside reverse transcriptase inhibitors (NNRTIs), nucleoside reverse transcriptase inhibitors (NRTIs), analogs, variants etc.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “eradication” of a retrovirus, e.g. human immunodeficiency virus (HIV), as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this disclosure “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the disclosure find use in experimental animals, in veterinary application, and in the development of animal models for disease, including, but not limited to, rodents including mice, rats, and hamsters, and primates.

The term “percent sequence identity” or having “a sequence identity” refers to the degree of identity between any given query sequence and a subject sequence.

As used herein, a “pharmaceutically acceptable” component/carrier etc. is one that is suitable for use with humans and/or animals without undue adverse side effects (such as toxicity, irritation, and allergic response) commensurate with a reasonable benefit/risk ratio.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

To “treat” a disease as the term is used herein, means to reduce the frequency or severity of at least one sign or symptom of a disease or disorder experienced by a subject. Treatment of a disease or disorders includes the eradication of a virus.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof; or (4) relieving the disease, i.e., causing regression of the state, disorder or condition or at least one of its clinical or subclinical symptoms. The benefit to an individual to be treated is either statistically significant or at least perceptible to the patient or to the physician.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the disclosure can include a single treatment or a series of treatments.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

FIGS. 1A-1K are a series of schematics, blots and grapshs demonstrating the gRNA locations, CCR5 excision, and viral and immune profiles from ART and CRISPR-Cas9 treatments of HIV-1-infected humanized mice. FIG. 1A: Chromosomal location and coordinates of CRISPR gRNA target sequences in the human (′(′R5 gene. The (′(′R5 coding sequence (CDS) is highlighted in yellow, positions of start and stop codons are in the red, the position of D32 mutation is shown as a patterned box, gRNA target sequences are highlighted in green, and PAMs in red. FIG. 1B: Agarose gel analysis of PCR genotyping of CRISPR-Cas9 mediated cleavage of CCR5 gene. Genomic DNAs from two control (CTRL1 and CTRL2) and two CCR5 knockout (C14 and C18) TZM-bl single cell clones were used as PCR template. FIG. 1C: Alignment of Sanger sequencing results confirming CRISPR-induced truncation of CCR5. FIG. 1D: CCR5 mRNA expression in knockout clones was checked by reverse transcription-qPCR from two controls and two knockout clones. FIG. 1E: Schematic illustration to look for CCR5 expression in human immune cells at different time points after a single IV injection of AAV6-CRISPR-Cas9 targeting CCR5 gene, in healthy humanized mice. FIG. 1F: Flow cytometric analysis of peripheral blood examined presence of CD3+CD4+CCR5+ T cells at days 0, 3, 5 and 7 days after a single injection. Data are expressed as mean±SEM. FIG. 1G: CCR5 excision analysis on blood cells after single AAV₆-CRISPR-Cas9 injection on the same humanized mice at different time points as described in FIGS. 1E-1F. The full-length and truncated bands are pointed in arrows. FIG. 1H: Sanger sequencing of the bands from humanized mice from F, confirming CRISPR induced truncation of CCR5 gene. FIG. 1I: Study scheme showing the timing of NSG-humanized mice generation, HIV-1 infection, ART and single or dual CRISPR treatments to respective groups. After 2 weeks of infection and confirmation of VL, mice were given intramuscular (IM) doses with 45 mg/kg NMCAB and NRPV and 40 mg/kg NM3TC, NMABC. Treatment was for 4 weeks, followed by a single IV dose of AAV₆-CRISPR-Cas9 CCR5, at week 7 followed by a second IV dose of AAV₉-CRISPR-Cas9 LTR-Gag at week 8. Animals had antiretroviral medicines stopped for eleven weeks at the time of sacrifice. FIG. 1J: Evaluation of human CD45+CD3+CD4⁺ T cell numbers in humanized mice by flow cytometry tests on 0, 2, 7, 8, 12- and 17-weeks post-infection. FIG. 1K: Plasma viral load assessment by determining viral RNA copies of individual animals assayed at 2, 7, 8, 12 and 17 weeks after HIV-1 NL_{ADA} infection from the LA-ART and dual CRISPR treatment group using COBAS Ampliprep-Taqman-48 V 2.0 assay with a sensitivity of detection at 200 copies/ml after adjustment. One animal represented in green showed viral RNA at the detection limit at study end.

FIGS. 2A-2E are a series of blots, graphs and photographs of RNAscope assays of tissues demonstrating the viral elimination in HIV-1 infected CRISPR-treated humanized mice. FIG. 2A: HIV-1 DNA detection was made by semi-nested real-time qPCR assays. Analyses were performed from spleen, bone marrow, gut, brain, liver, and lung tissues in each of the treatment groups. The data are expressed as total HIV-1 DNA copies/10⁶ human CD45+ cells. Six of ten animals with dual CRISPR treatments, 2/7 in ART and LTR-Gag CRISPR, and ⅙ in ART and CCR5 CRISPR showed complete viral elimination from all analyzed tissues. FIG. 2B: RNAscope assay for detecting HIV-1 RNA revealed single brown dots or cluster of dots in 5 μm thick spleen sections using antisense probe V-HIV1-Clade-B (ACD cat #416111) targeting 854-8291 base pair of HIV-1. Representative ART and dual CRISPR mouse spleens showing no signals corresponding to the presence of viral RNA, the right most spleen from one mouse demonstrating viral rebound at study's end. Human peptidylprolyl Isomerase B was used as positive control for every tissue analyzed. Images are 20× magnification. FIG. 2C: Ultrasensitive ddPCR with sensitivity of detecting 1-2 copies was used to detect viral DNA in the various organs of the ten mice with ART and dual CRISPR treatments. Note that the six animals with double CRISPR treatment showed complete elimination of virus in all the tested tissues. FIG. 2D: An VOA was performed by adoptive transfer of splenocytes and BM cells from ART and dual CRISPR-Cas9 treated 10 animals to uninfected recipient CD34+NSG-humanized mice. Cells isolated from same six animals failed to show viral recovery after five weeks of examination by plasma viral RNA measurements as shown in green circles and boxes and used as the definition for viral eradication. FIG. 2E: CCR5 and HIV-1 excision study on spleen post-ART and dual CRISPR-Cas9 injection in infected humanized mice. Total DNA from spleen from all ten dual treated animals are used for PCR genotyping with primer sets derived from the 5′LTR, 3′LTR, and the HIV-1 gag and CCR5 gene. Top-panel shows excision of CCR5 DNA in HIV-1 infected humanized mice, as the CCR5 expression came back to normal, we didn't observe the excised band, except in one mouse. Middle-panel shows excision of HIV-1 DNA from 3′LTR to Gag by CRISPR-Cas9 in HIV-1 infected humanized mice and bottom-panel shows CRISPR-Cas9 excision of HIV-1 Gag to 5′LTR in infected humanized mice.

FIGS. 3A, 3B are a series of plots and graphs demonstrating that knockout of CCR5 gene expression protects cells from infection by CCR5-tropic HIV-1. FIG. 3A: Immunolabelling/flow cytometry analysis of CCR5 antigen expression in control (CTRL1 and CTRL2) and CCR5 knockout (C14 and C18) TZM-bl cells. FIG. 3B: Control and CCR5 knockout cells were infected with CCR5-tropic HIV-1 NL4-3-BAL-GFP (top panel) or pan-tropic HIV-1 NL4-3-GFP-P2A-Nef/VSV-g (bottom panel) at different multiplicities of infection (MOIs, from 0.01 to 1). 48 h later, GFP expression was measured by flow cytometry on paraformaldehyde-fixed cells. As expected, CCR5 knockout cells (C14 and C18) were resistant to infection with CCR5-tropic but not to infection with pan-tropic, VSV-g pseudotyped HIV-1.

FIG. 4 is a series of plots demonstrating CCR5 expression in immunocytes of humanized mice. CCR5 expression from CD3″CD4″ T cells from a representative hu-mouse injected “treated” with AAV6 CCR5 CRISPR-Cas9 excision and one control. Mice were bled prior to and 3, 5 and 7 days after injection. CCR5 expression was diminished for up to 7 days in the AAV6 CCR5 CRISPR-Cas9 treated mice compared to untreated controls. CCR5 CRISPR-Cas9 was administered to a group of 5 mice through AAV6 delivery by a tail vein intravenous injection. A replicate group of 5 untreated hu-mice served as controls.

FIGS. 5A1-5C are a series of graphs demonstrating the Plasma viral load of ART and CRISPR CCR5-HIV-1 treatments of infected hu-mice. FIGS. 5A1-5A3: Plasma HIV-1 RNA copies in untreated control hu-mice (n=8, shown in red), HIV-1_{ADA} infected ART treated (n=9, shown in blue) and infected and ART and AAV6-CRISPR-Cas9 CCR5 and AAV9 CRISPR-Cas9 HIV-1 LTR Gag genes (n=8, shown in black line). Each of the treatments followed two-weeks of virus infection of hu-mice. Viral rebound was observed at study end in all 9 animals after ART withdrawal. This was also seen in select animals in the CRISPR treated groups. The data represent mean±SEM for each group. FIG. 5B: Plasma viral load of individual animals (n=6) treated with LA-ART and AAV6 CCR5 CRISPR-Cas9. Five of 6 hu-mice showed viral rebound at 17 weeks following viral infection. FIG. 5C: Plasma viral load of individual animals (n=7) treated with ART and CRISPR-Cas9 targeting HIV-1 LTR Gag. Five of 7 animals showed viral rebound. Plasma viral load of the individual animal groups was assayed at 2, 7, 8, 13 and 17 weeks after viral infection. HIV-1 RNA was determined by the COBAS Ampliprep-Taqman-48 V 2.0 assay. The sensitivity of detection for the mice were at 200 copies/ml after adjustments for plasma dilution.

FIG. 6 is a series of graphs demonstrating the viral RNA in HIV-1 infected and ART-CRISPR treated hu-mouse tissues. HIV-1 RNA levels were measured in the gag region of HIV-1 using ultrasensitive semi-nested real-time qPCR assays. These tests were performed in spleen, lung, gut, bone marrow, brain, and liver from each of the treatment groups. The data represent the following: HIV-1 infected (n=8), HIV-1 infected and dual CRISPR-Cas9 treated (n=8), HIV-1 infected and ART treated (n=9), HIV-1 infected and ART treated and CRISPR CCR5 (n=6), HIV-1 infected ART treated and CRISPR LTR Gag (n=7) and HIV-1 infected and ART and CRISPR CCR5 and LTR Gag treated mice (n=10). One of six animals from ART and CRISPR CCR5 group, two out of seven mice from ART and CRISPR HIV-1 LTR Gag group and six out of ten animals from ART and CRISPR CCR5 and HIV-1 LTR Gag group demonstrated viral elimination. The data are shown in green open boxes. For these hu-mice viral amplification assay failed to demonstrate viral nucleic acid. The detection limit of the assay is 10 copies of viral RNA. The data are expressed as total HIV-1 RNA copies/10⁶ human CD45+ cells. The data represent mean±SEM for each group.

FIGS. 7A, 7B are a series of graphs demonstrating the Viral DNA and RNA in HIV-1 infected and CRISPR treated hu-mice. HIV-1 DNA (FIG. 7A) and RNA (FIG. 7B) analyses in the gag region using ultrasensitive semi-nested real-time qPCR assays from kidney tissues. The data sets are shown from different treatment groups employed. Each plate represents the six individual groups. These include HIV-1 infected (n=8), HIV-1 infected and CRISPR treated (n=8), HIV-1 infected and ART treated (n=9), HIV-1 infected, ART treated and CRISPR targeting CCR5 (n=6), HIV-1 infected, ART treated and CRISPR HIV-1 LTR Gag (n=7) and HIV-1 infected, ART treated and CCR5 and HIV-1 LTR Gag CRISPR mice (n=10). One out of six animals from ART and CRISPR CCR5 and two out of seven animals from ART and CRISPR HIV-1 LTR Gag group and six out of ten animals in the ART and CRISPR CCR5 and HIV-1 LTR Gag groups failed to demonstrate virus. These are shown in green open boxes and showed no viral amplification. The detection limit of the assay is 10 copies of viral DNA/RNA. The data are expressed as total HIV-1 DNA/RNA copies/10⁶ human CD45⁺ cells. The data represent mean±SEM for each group.

FIG. 8 is a series of photographs of results from RNAscope assays. Representative results from RNAscope assay revealed the detection of single or clusters of brown dots corresponding to HIV-1 RNA in 5 μm-thick spleen sections of infected animals receiving ART and CRISPR targeting CCR5 and HIV-1 LTR Gag. Top panel shows HIV-1 infected animals treated with ART and AAV9 mediated CRISPR targeting HIV-1 LTR Gag. Here viral rebound was demonstrated in 5/7 animals after therapeutic cessation. Bottom panel shows hu-mice infected with HIV-1, treated with ART and CRISPR CCR5; 1/6 had no detectable HIV-RNA, in this group. The figures are representative tissue sections taken from each of the animal groups. In these assays, we used the antisense V-HIV1-Clade-B targeting 854-8291 bp of HIV-1 as the probe. Images were captured at 20× magnification. Human peptidyl Isomerase B was used as a positive control for the analyzed tissues.

FIG. 9 is a series of graphs demonstrating viral RNA in HIV-1 infected and CRISPR treated hu-mice tissues. HIV-1 RNA in the viral polymerase region using the ddPCR assay from spleen, lung, gut, brain, liver and kidney tissues from described treatment groups. The data represent each of the six groups HIV-1 infected (n=5), HIV-1 infected and CRISPR CCR5-HIV-1 treated (n=5), HIV-1 infected and ART treated (n=6), HIV-1 infected and ART treated and CRISPR CCR5 (n=6), HIV-1 infected, ART and CRISPR HIV-1 LTR Gag (n=7) treated, and HIV-1 infected and ART and CRISPR CCR5 and HIV-1 LTR Gag treated mice (n=10). The detection limit of the assay is 2 copies of viral RNA. The data are expressed as total HIV-1 RNA copies/106 human CD45⁺ cells. The data represent mean±SEM for each group.

FIGS. 10A, 10B are a series of plots demonstrating viral DNA in HIV-1 infected and CRISPR treated hu-mice tissues. HIV-1 DNA analyses in the HIV-1 Gag region using the ddPCR assay in spleen, lung, gut, brain, liver and kidney tissues from different treatment groups as described in FIGS. 5A1-5C. The data represent each of the two groups. (FIG. 10A) HIV-1 infected ART and CRISPR LTR Gag treated (n=7) and (FIG. 10B) is HIV-1 infected, ART and CRISPR CCR5 treated (n=6) (FIG. 10B). The detection limit of the assay is 2 copies of viral DNA. One/six animals from ART+CRISPR (CCR5) group and 2/7 animals from LA-ART+CRISPR (LTR-Gag) group showed no amplification of virus from all the tissues analyzed. The data are expressed as total HIV-1 DNA copies/10⁶ human CD45⁺ cells.

FIG. 11 is a series of plots demonstrating the results obtained from a viral outgrowth assay. An in vivo VOA was performed by adoptive transfer of splenocytes and BM cells from infected and ART treated and dual CRISPR administered all 10 animals to uninfected healthy new recipient CD34″ humanized mice. Six animals failed to show viral recovery after five weeks of examination of HIV-1 DNA from all the recipient mice as measured using real-time semi-nested qPCR. Detection of HIV-1 DNA in all the tissues in spleen (top panel) and bone marrow (bottom panel) of adoptively transferred humanized mice. The intent was to perform cross disciplinary viral amplification from known infectious cell reservoirs. HIV-1 DNA analyses using ultrasensitive semi-nested real-time qPCR assay was performed from spleen, BM, lung, gut, liver, kidney and brain tissues of adoptively transferred hu-mice, 5 weeks post-transfer. The data are expressed as total HIV-1 DNA/10⁶ human CD45⁺ cells. Six animals shown under dashed line in black dots had no viral recovery. The data confirm complete elimination of virus from these tissues analyzed that showed no evidence of viral rebound from the donor cells isolated from ART and dual CRISPR treated animals. The detection limit of assay is 10 copies. The data are expressed as total HIV-1 DNA copies/10⁶ human CD45⁺ cells. The data represent mean±SEM for each group.

FIGS. 12A, 12B are a series of plots demonstrating CCR5 and CXCR4 expression in spleen. HIV-1 DNA analyses of CCR5 (FIG. 12A) and CXCR4 (FIG. 12B) expression using ultrasensitive semi-nested real-time qPCR assay from spleen tissues of individual animals from four treatment groups at the study end. This confirms the presence of abundant CCR5 and CXCR4 expression. The red filled boxes in the dual treatment group are the animals with LA-ART and dual CRISPR treated with no HIV-1 amplification from previous figures and had the CCR5 expression restored. The data are expressed as total HIV-1 DNA copies/10⁶ human CD45⁺ cells. The data represent mean±SEM for each group.

FIGS. 13A-13C are a series of blots demonstrating CCR5 and HIV-1 excision in brains of infected and treated hu-mice. CCR5 and HIV-1 excision study was performed on brain post-LA-ART with or without a single or dual CRISPR-Cas9 injection in infected humanized mice. Total DNA from brain of all untreated/treated animals is used for PCR genotyping with primer sets derived from the 5′LTR, 3′LTR, and the HIV-1 gag and CCR5 gene. (FIG. 13A) shows excision of CCR5 DNA in HIV-1 infected humanized mice, as the CCR5 expression came back to normal, we didn't observe the excised band, except in one mouse. (FIG. 13B) shows excision of HIV-1 DNA from 3′LTR to Gag by CRISPR-Cas9 in HIV-1 infected humanized mice and (FIG. 13C) shows CRISPR-Cas9 excision of HIV-1 Gag to 5′LTR in infected humanized mice.

FIGS. 14A, 14B are a series of blots demonstrating CCR5 excision in lung and liver of infected and treated humanized mice. CCR5 excision study on lung (FIG. 14A) and liver (FIG. 14B) tissues post-LA-ART and without or single or dual CRISPR-Cas9 injection in infected humanized mice. Total DNA from lung and livers from all untreated/treated animals is used for PCR genotyping with primer sets for CCR5 gene. Panel shows no excision band of CCR5 DNA in HIV-1 infected humanized mice, as the CCR5 expression came back to normal at study end.

FIG. 15 is a series of schematics showing a summary of HIV-1 excision analysis. The presence of CRISPR-Cas9-mediated excision of proviral sequences in tissues of AAV₉-CRISPR-HIV treated animals were checked by PCR-genotyping followed by verification of detected CRISPR-cleaved/end-joined truncated amplicons by Sanger sequencing. Two CRISPR-Cas9-mediated excision events were investigated: 5′LTR-gag (4978 bp) and gag-3′LTR (Δ8097 bp). Squares represent tested tissue samples. Tissues that were not available for analysis are shown as empty spaces. The animals showing a lack of viral rebound (undetectable plasma viral RNA) are marked red (11 out of 19, =58%). One animal from the first set that received ART and dual CRISPR-Cas9, #392 (marked with a black star), had a very low 400 HIV-1 RNA copies/ml in plasma at 17WPI but no evidence of viral RNA (RNAscope) or DNA (qPCRs and ddPCRs) in the examined tissues. Another animal from the second set of ART and dual CRISPR-Cas9 treated animals, #712 (marked with a red star), had undetectable plasma viral RNA but tested positive for viral DNA in tissues using ddPCR.

FIGS. 16A-16C are a series of blots demonstrating an HIV excision study in brain, gut and liver of infected and treated hu-mice. HIV excision study on Brain (FIG. 16A), gut (FIG. 16B) and liver (FIG. 16C) tissues ART and with single or dual CRISPR-Cas9 treatments in infected hu-mice. Total DNA from brain, gut and liver from all single or dual treated animals are used for PCR genotyping with primers sets derived from the 5′LTR, 3′LTR, and the HIV-1 gag gene. The figure shows excision of HIV-1 DNA from 3′LTR to Gag by CRISPR-Cas9 in HIV-1 infected humanized mice and CRISPR-Cas9 excision of HIV-1 Gag to 5′LTR in infected humanized mice. The excised band for 5′LTR-gag is 193 bp and for gag-3′LTR is 523 bp as highlighted.

FIG. 17 is a graph showing a plasma viral load of validation study. Plasma HIV-1 RNA copies of individual dual treated animals (n=9) treated with LA-ART and two sequential treatments of CRISPR-Cas9 targeting CCR5 gene of host and LTR-Gag of HIV-1 in infected humanized mice. Evaluation of plasma viral load indicated that after sequential administration of both CRISPR-Cas9, 6 out of 9 mice showed no evidence of viral rebound in the plasma at 17 weeks (study end). Plasma viral load of individual animals was assayed at 2, 7, 8, 13- and 17-weeks post-infection for HIV-1 RNA as determined using COBAS Ampliprep-Taqman-48 V2.0 assay kit with a sensitivity of detection at 140 copies/ml after dilution factor adjustment.

FIG. 18 is a graph demonstrating flow cytometric evaluations of human CD4⁺ T cells in hu-mice. Peripheral blood of CD34⁺ NSG-humanized mice was assayed before (0) and 2, 7, 8, 13 and 17 weeks post-infection with HIV-1_{ADA} for human CD4⁺ cells from CD45⁺CD3⁺ gated populations. These experiments assessed CD4⁺ T cells patterns throughout the course of the study. The percentage of human CD4⁺ T cells followed a decreasing pattern in all three mice in the HIV-1_{ADA} infected group. CD4⁺ T cell profile of HIV-1 and ART treated and dual CCR5-HIV-1 CRISPR administered showed restoration of absolute numbers of CD4+ T cells at the study end which was 17 weeks after infection.

FIG. 19 is a plot demonstrating viral DNA in HIV-1 infected and treated humanized mice tissues. HIV-1 DNA analyses in the gag region using ultrasensitive semi-nested real-time qPCR assays from spleen, lung, gut, bone marrow, brain, kidney, reproductive organ and liver from dual treatment group as described in FIG. 18. The data represent all the mice from HIV-1 infected and LA-ART treated and dual CRISPR CCR5 and HIV-1 LTR Gag (n=9). Five/nine animals (numbers 706, 709, 622, 651, 674) shown in green open boxes in the dual treatment group showed no amplification of virus from all the tissues analyzed. The detection limit of the assay is 10 copies of viral RNA. One mouse (number 712) which was undetectable in plasma viremia found to be HIV″ in tissue PCRs as shown in red, highlighting the importance of tissue reservoirs and use of sensitive assays for the detection. The other 3 rebound mice (numbers 705, 707 and 713) were found to be highly positive. The data are expressed as total HIV-1 RNA copies/10⁶ human CD45⁺ cells. The data represent mean±SEM for each group.

FIG. 20 is a series of photographs demonstrating results obtain from RNAscope assays. Representative results from RNAscope assay revealed the detection of single or clusters of brown dots corresponding to HIV-1 RNA in 5 μm-thick spleen sections of infected animals receiving LA-ART and CRISPR-Cas9 targeting CCR5 mediated by AAV6 and CRISPR-Cas9 mediated by AAV9 targeting LTR-Gag. 5 animals (numbers 706, 709, 622, 651, 674) failed to demonstrate viral RNA amplification by RNAscope assays. The right panel shows one mouse spleen with viral rebound at the study end. The figures are representative tissue sections taken from the dual animal group. In these assays, we used the antisense V-HIV1-Clade-B targeting 854-8291 bp of HIV-1 as the probe. Images were captured at 40× magnification. Human peptidyl Isomerase B was used as a positive control for all tissues analyzed.

FIG. 21 is a plot demonstrating detection of HIV-1 DNA by ddPCR. Ultrasensitive ddPCR with sensitivity of detecting 1-2 copies was used to detect viral DNA in the various organs (spleen, lung, liver, kidney, gut and bone marrow, reproductive organ and brain) of all 9 mice with ART and dual CRISPR treatments. Note that the five animals with double CRISPR treatment showed complete elimination of virus in all the tissues tested. One mouse (#712) which was undetectable in plasma viremia found to be HIV-1 positive in tissue PCRs as shown in red open triangle, highlighting tissue reservoirs and use of sensitive assays for the detection. These results provide evidence of viral elimination in the five mice (#706, 709, 622, 651, 674) that showed no evidence for rebound after ART cessation and had no viral DNA after CRISPR-Cas9 treatment.

FIG. 22 is a plot demonstrating a viral outgrowth assay to detect replication-competent virus in dual treated humanized mice. The assay was performed by adoptive transfer of splenocytes and BM cells (˜8-10×10⁶ cells/mice/tissues) from ART and dual CRISPR-Cas9 treated animals (7 humanized mice) to uninfected recipient CD34 NSG-humanized mice (14 mice, 7 received splenocytes and other 7 received BM cells). Cells isolated from same virally suppressed animals failed to show viral recovery after five weeks of examination by plasma viral RNA measurements as shown in green circles and boxes and used as the definition for viral eradication.

FIGS. 23A, 23B are a series of plots demonstrating the detection of HIV-1 DNA in tissues in adoptively transferred humanized mice. Splenocytes and bone marrow (BM) cells were isolated from HIV-1 infected mice with prior ART and dual CRISPR treatment and used for adoptive transfers into new CD34″ NSG-humanized mice. The intent was to perform cross disciplinary viral amplification from known infectious cell reservoirs. FIG. 23A: HIV-1 DNA analyses using semi-nested real-time qPCR assays from spleen, bone marrow and lung tissues of adoptively transferred humanized mice. The data are expressed as total HIV-1 DNA copies/10⁶ human CD45⁺ cells. Three animals shown by green circles and squares below dotted line), showed no viral recovery. The above data were further confirmed using the ddPCR assay (FIG. 23B), where the adoptively transferred recipient animals failed to demonstrate HIV-1 products indicating complete viral elimination. Virus was recovered from all rebound mouse tissues. The data are expressed as total HIV-1 DNA copies/microgram of DNA used for ddPCR assay after normalization to human cells. The blue color dot mice were negative in semi-nested real-time qPCR but were found to be positive in ddPCR, highlighting the importance of using a sensitive assay (detection limit of 1-2 copies) for viral detection. The detection limit for real-time qPCR is 10 copies and is 1 copy for ddPCR. The data represent mean±SEM for each group.

FIG. 24 is a series of photographs of cytostains demonstrating liver, kidney and spleen tissue histology in treated hu-mice. Hematoxylin and eosin staining of representative sections from liver, kidney and spleen tissues in HIV-1_{ADA}-infected, ART treated and dual CRISPR injected humanized mice at the endpoint of the study. Tissue pathology was not observed in ART alone or dual treatment groups as compared to untreated controls. The images were captured at 20-× magnification.

FIG. 25 is a series of schematics demonstrating that while in LA-ART-only treated animals at 17WPI, plasma viral loads were consistently higher (5×-1000×) than pre-LA-ART setpoint (2WPI), in LA-ART+CRISPR-CCR5 and LA-ART+CRISPR-HIV treatment groups majority of animals (67% and 71% respectively) showed lower than pre-LASER-ART plasma viral loads in addition to animals with lack of viral rebound.

### DETAILED DESCRIPTION

Embodiments of the disclosure are directed to compositions that eliminate retrovirus genomes form an infected cell and the prevention of further infection by interfering with receptor expression or function that the virus uses to infect a cell. Compositions include the use of RNA-guided Clustered Regularly Interspace Short Palindromic Repeat (CRISPR)-Cas nuclease systems (Cas/gRNA) in single and multiplex configurations that target the retroviral genome as well as the genes encoding receptors used by the virus to infect a cell.

### Compositions

The compositions disclosed herein may include nucleic acids encoding a CRISPR-associated endonuclease, such as Cas9. In some embodiments, one or more guide RNAs that are complementary to a target sequence of HIV may also be encoded. Accordingly, in some embodiments composition for use in inactivating a proviral DNA integrated into the genome of a host cell infected, including a latent infection, with human immunodeficiency virus (HIV), the composition comprises at least one isolated nucleic acid sequence encoding a Clustered Regularly Interspaced Short Palindromic Repeat (CRISPR)-associated endonuclease, and at least one guide RNA (gRNA), said at least one gRNA having a spacer sequence that is complementary to one or more target sequences, wherein the target sequences comprise nucleic acids in a chemokine receptor, in a long terminal repeat (LTR), in a Gag sequence of a proviral HIV DNA. In certain embodiments, the at least one gRNA comprises a nucleic acid sequence complementary to a target nucleic acid sequence having a sequence identity of at least 75% to one or more target nucleic acid sequences, fragments, mutants, variants or combinations thereof. In other embodiments, the at least one gRNA comprises at least one nucleic acid sequence complementary to a target nucleic acid sequence.

The isolated nucleic acid can be encoded by a vector or encompassed in one or more delivery vehicles and formulations as described in detail below.

### CRISPR-Cas Systems

The CRISPR-Cas system includes a gene editing complex comprising a CRISPR-associated nuclease, e.g., Cas9, and a guide RNA complementary to a target sequence situated on a DNA strand, such as a target sequence in proviral DNA integrated into a mammalian genome, a receptor used by a virus to infect a cell, e.g. HIV and CCR5 receptor. The gene editing complex can cleave the DNA within the target sequence. This cleavage can in turn cause the introduction of various mutations into the proviral DNA, resulting in inactivation of HIV provirus. The mechanism by which such mutations inactivate the provirus can vary. For example, the mutation can affect proviral replication, and viral gene expression. The mutations may be located in regulatory sequences or structural gene sequences and result in defective production of HIV. The mutation can comprise a deletion. The size of the deletion can vary from a single nucleotide base pair to about 10,000 base pairs. In some embodiments, the deletion can include all or substantially all of the integrated retroviral DNA sequence. In some embodiments the deletion can include the entire integrated retroviral DNA sequence. The mutation can comprise an insertion, that is, the addition of one or more nucleotide base pairs to the pro-viral sequence. The size of the inserted sequence also may vary, for example from about one base pair to about 300 nucleotide base pairs. The mutation can comprise a point mutation, that is, the replacement of a single nucleotide with another nucleotide. Useful point mutations are those that have functional consequences, for example, mutations that result in the conversion of an amino acid codon into a termination codon or that result in the production of a nonfunctional protein.

In embodiments, the CRISPR/Cas system can be a type I, a type II, or a type III system. Non-limiting examples of suitable CRISPR/Cas proteins include Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In addition to the wild type and variant Cas9 endonucleases described, embodiments of the disclosure also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI:10.1126/science.aad5227).

In certain embodiments, three variants found to have the best cleavage efficiency and fewest off-target effects: SpCas9 (K855A), SpCas9(K810A/K1003A/R1060A) (a.k.a. eSpCas9 1.0), and SpCas9(K848A/K1003A/R1060A) (a.k.a. eSPCas9 1.1) are employed in the compositions. The disclosure is by no means limited to these variants, and also encompasses all Cas9 variants (Slaymaker, I. M. et al. Science. 2016 Jan. 1; 351(6268):84-8. doi: 10.1126/science.aad5227. Epub 2015 Dec. 1). The present disclosure also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D1135E), SpCas9-HF3 (N497A/R661A/Q695A/Q926A/L169A), SpCas9-HF4 (N497A/R661A/Q695A/Q926A/Y450A). Also included are all SpCas9 variants bearing all possible single, double, triple and quadruple combinations of N497A, R661A, Q695A, Q926A or any other substitutions (Kleinstiver, B. P. et al., 2016, Nature. DOI: 10.1038/nature16526).

As used herein, the term “Cas” is meant to include all Cas molecules comprising variants, mutants, orthologues, high-fidelity variants and the like.

In one embodiment, the endonuclease is derived from a type II CRISPR/Cas system. In other embodiments, the endonuclease is derived from a Cas9 protein and includes Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Included are Cas9 proteins encoded in genomes of the nanoarchaea ARMAN-1 (Candidatus Micrarchaeum acidiphilum ARMAN-1) and ARMAN-4 (Candidatus Parvarchaeum acidiphilum ARMAN-4), CasY (Kerfeldbacteria, Vogelbacteria, Komeilibacteria, Katanobacteria), CasX (Planctomycetes, Deltaproteobacteria).

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains. Active DNA-targeting CRISPR-Cas systems use 2 to 4 nucleotide protospacer-adjacent motifs (PAMs) located next to target sequences for self versus non-self discrimination. ARMAN-1 has a strong ‘NGG’ PAM preference. Cas9 also employs two separate transcripts, CRISPR RNA (crRNA) and trans-activating CRISPR RNA (tracrRNA), for RNA-guided DNA cleavage. Putative tracrRNA was identified in the vicinity of both ARMAN-1 and ARMAN-4 CRISPR-Cas9 systems (Burstein, D. et al. New CRISPR-Cas systems from uncultivated microbes. Nature. 2017 Feb. 9; 542(7640):237-241. doi: 10.1038/nature21059. Epub 2016 Dec. 22).

Embodiments of the disclosure also include a new type of class 2 CRISPR-Cas system found in the genomes of two bacteria recovered from groundwater and sediment samples. This system includes Cas1, Cas2, Cas4 and an approximately ˜ 980 amino acid protein that is referred to as CasX. The high conservation (68% protein sequence identity) of this protein in two organisms belonging to different phyla, Deltaproteobacteria and Planctomycetes, suggests a recent cross-phyla transfer. The CRISPR arrays associated with each CasX has highly similar repeats (86% identity) of 37 nucleotides (nt), spacers of 33-34 nt, and a putative tracrRNA between the Cas operon and the CRISPR array. Distant homology detection and protein modeling identified a RuvC domain near the CasX C-terminal end, with organization reminiscent of that found in type V CRISPR-Cas systems. The rest of the CasX protein (630 N-terminal amino acids) showed no detectable similarity to any known protein, suggesting this is a novel class 2 effector. The combination of tracrRNA and separate Cas1, Cas2 and Cas4 proteins is unique among type V systems, and phylogenetic analyses indicate that the Cas1 from the CRISPR-CasX system is distant from those of any other known type V. Further, CasX is considerably smaller than any known type V proteins: 980 aa compared to a typical size of about 1,200 amino acids for Cpf1, C2c1 and C2c3 (Burstein, D. et al., 2017 supra).

Another new class 2 Cas protein is encoded in the genomes of certain candidate phyla radiation (CPR) bacteria. This approximately 1,200 amino acid Cas protein, termed CasY, appears to be part of a minimal CRISPR-Cas system that includes Cas1 and a CRISPR array. Most of the CRISPR arrays have unusually short spacers of 17-19 nt, but one system, which lacks Cas1 (CasY.5), has longer spacers (27-29 nt). Accordingly, in some embodiments of the disclosure, the CasY molecules comprise CasY.1, CasY.2, CasY.3, CasY.4, CasY.5, CasY.6, mutants, variants, analogs or fragments thereof.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas protein or fragment thereof. In other embodiments, the CRISPR/Cas-like protein can be derived from modified Cas proteins. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In some embodiments, the CRISPR-associated endonuclease can be a sequence from another species, for example, other bacterial species, bacteria genomes and archaea, or other prokaryotic microorganisms. Alternatively, the wild type Cas9, CasX, CasY.1, CasY.2, CasY.3, CasY.4, CasY.5, CasY.6, ARMAN 1, ARMAN 4, sequences can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in GENBANK accession numbers KM099231.1 GI:669193757; KM099232.1 GI:669193761; or KM099233.1 GI:669193765. Alternatively, the Cas9, CasX, CasY.1, CasY.2, CasY.3, CasY.4, CasY.5, CasY.6, ARMAN 1, ARMAN 4, sequences can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of GENBANK accession numbers KM099231.1 GI:669193757; KM099232.1 GI:669193761; or KM099233.1 GI:669193765, or Cas9 amino acid sequence of PX330 or PX260 (Addgene, Cambridge, MA).

The wild type Cas9, CasX, CasY.1, CasY.2, CasY.3, CasY.4, CasY.5, CasY.6, ARMAN 1, ARMAN 4, sequences can be a mutated sequence. For example, the Cas9 nuclease can be mutated in the conserved HNH and RuvC domains, which are involved in strand specific cleavage. In another example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. The sequences of Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof, can be modified to encode biologically active variants, and these variants can have or can include, for example, an amino acid sequence that differs from a wild type by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, polypeptides can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9, CasX, CasY.1, CasY.2, CasY.3, CasY.4, CasY.5, CasY.6, spCas, eSpCas, SpCas9, ARMAN 1, ARMAN 4 polypeptides. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

Two nucleic acids or the polypeptides they encode may be described as having a certain degree of identity to one another. For example, a Cas9 protein and a biologically active variant thereof may be described as exhibiting a certain degree of identity. Alignments may be assembled by locating short Cas9 sequences in the Protein Information Research (PIR) site (pir.georgetown.edu), followed by analysis with the “short nearly identical sequences” Basic Local Alignment Search Tool (BLAST) algorithm on the NCBI website (ncbi.nlm.nih.gov/blast).

A percent sequence identity to Cas9 can be determined and the identified variants may be utilized as a CRISPR-associated endonuclease and/or assayed for their efficacy as a pharmaceutical composition. A naturally occurring Cas9 can be the query sequence and a fragment of a Cas9 protein can be the subject sequence. Similarly, a fragment of a Cas9 protein can be the query sequence and a biologically active variant thereof can be the subject sequence. To determine sequence identity, a query nucleic acid or amino acid sequence can be aligned to one or more subject nucleic acid or amino acid sequences, respectively, using the computer program ClustalW (version 1.83, default parameters), which allows alignments of nucleic acid or protein sequences to be carried out across their entire length (global alignment). See Chenna et al., Nucleic Acids Res. 31:3497-3500, 2003.

In some embodiments, the isolated nucleic acids sequences can be encoded by the same construct with one or more isolated nucleic acids sequences directed toward a first and second retroviral target sequence, and one or more isolated nucleic acids sequences directed toward a one or more target sequences of one or more receptors that a virus uses to infect a cell, e.g. in the case of HIV, the receptor can be CCR5, CD4 or CXCR4.

In some embodiments, the one or more isolated nucleic acids sequences are encoded by two or more constructs with one member directed toward a first retroviral target sequence, and the other member toward a second retroviral target sequence excises or eradicates the retroviral genome from an infected cell. Another construct is directed to a receptor that a virus uses to infect a cell, e.g. in the case of HIV, the receptor can be CCR5, CD4 or CXCR4.

Accordingly, the disclosure features compositions for use in inactivating a proviral DNA integrated into a host cell, including an isolated nucleic acid sequence encoding a CRISPR-associated endonuclease and one or more isolated nucleic acid sequences encoding one or more gRNAs complementary to a target sequence in HIV or another retrovirus. A second isolated nucleic acid sequence encoding a CRISPR-associated endonuclease and one or more isolated nucleic acid sequences encoding one or more gRNAs complementary to a target sequence encoding a receptor used by a virus to infect a cell. The isolated nucleic acid can include one gRNA, two gRNAs, three gRNAs etc. Furthermore, the isolated nucleic acid can include one or more gRNAs complementary to target sequences in the retrovirus and a second isolated nucleic acid can include one or more gRNAs complementary to target sequences encoding receptors used by the virus to infect a cell. Alternatively, each isolated nucleic acid can include at least one gRNA complementary to a target virus sequence and at least one a gRNA complementary to target sequences encoding receptors used by the virus to infect a cell. One of ordinary skill in the art would only be limited by their imagination with respect to the various combinations of gRNAs.

In some embodiments, a composition for preventing or treating a retroviral infection in vitro or in vivo comprises at least two isolated nucleic acid sequences encoding: a first Clustered Regularly Interspaced Short Palindromic Repeat (CRISPR)-associated endonuclease and at least one guide RNA (gRNA), the gRNA being complementary to a target sequence in the integrated retroviral DNA; a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell in vitro or in vivo. In some embodiments, the endonuclease comprises Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments or combinations thereof. The endonucleases may be the same or may vary. For example, one endonuclease may be a Cas9, another endonuclease may be CasY.5 or ARMAN 4 and the like. Accordingly, the isolated nucleic acid sequence can encode any number and type of endonuclease.

In some embodiments, at least one gRNA is complementary to a target sequence in the integrated retroviral DNA and at least one gRNA is complementary to a target sequence in a gene encoding for at least one receptor used by a retrovirus for attachment and/or infection of a cell. In another embodiment, two or more gRNAs are complementary to two or more different target sequences in the integrated retroviral DNA and two or more guide RNAs (gRNAs), are complementary to two or more target sequences in a gene encoding for at least one receptor used by a retrovirus for attachment and/or infection of a cell in vitro or in vivo. In some embodiments, the isolated nucleic acid encodes at least one gRNA complementary to a target sequence in the integrated retroviral DNA and at least a first gRNA that is complementary to a first target sequence in a gene encoding for at least one receptor used by a retrovirus for attachment and/or infection of a cell; and a second gRNA that is complementary to a second target sequence in a gene encoding for at least one receptor used by a retrovirus for attachment and/or infection of a cell.

In some embodiments, the isolated nucleic acid encodes at least one gRNA complementary to a gene encoding at least one receptor used by a retrovirus for attachment and/or infection of a cell, and at least a first gRNA that is complementary to a first target sequence in the integrated retroviral DNA and at least a second gRNA that is complementary to a second target sequence in the integrated retroviral DNA. Accordingly, any number and combinations of gRNAs with different target sequences can be used to target desired target sequences.

In some embodiments, gRNA targets comprise one or more target sequences in an LTR region of an HIV proviral DNA and one or more targets in a structural gene of the HIV proviral DNA; or, one or more targets in a second gene; or, one or more targets in a first gene and one or more targets in a second gene; or, one or more targets in a first gene and one or more targets in a second gene and one or more targets in a third gene; or, one or more targets in a second gene and one or more targets in a third gene or fourth gene; or, any combinations thereof.

In some embodiments, gRNA targets comprise one or more target sequences in a gene encoding at least one receptor used by a retrovirus for attachment and/or infection of a cell and one or more targets in another gene associated with a viral infection; or, one or more targets in a second gene; or, one or more targets in a first gene and one or more targets in a second gene; or, one or more targets in a first gene and one or more targets in a second gene and one or more targets in a third gene; or, one or more targets in a second gene and one or more targets in a third gene or fourth gene; or, any combinations thereof.

In certain embodiments, a composition for preventing or treating a retroviral infection in vitro or in vivo, the composition comprises at least two isolated nucleic acid sequences wherein the first isolated nucleic acid sequences encodes a first Clustered Regularly Interspaced Short Palindromic Repeat (CRISPR)-associated endonuclease and at least one guide RNA (gRNA), the gRNA being complementary to a target sequence in the integrated retroviral DNA; the second isolated nucleic acid sequences encodes a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell in vitro or in vivo.

In certain embodiments, the first isolated nucleic acid sequences encodes at least one gRNA, the gRNA being complementary to a target sequence in the integrated retroviral DNA and a second gRNA that is complementary to a second target sequence in the integrated retroviral DNA. In certain embodiments, the second isolated nucleic acid sequence encodes a first gRNA that is complementary to a first target sequence in a gene encoding for at least one receptor used by a retrovirus for attachment and/or infection of a cell; and a second gRNA that is complementary to a second target sequence in a gene encoding for at least one receptor used by a retrovirus for attachment and/or infection of a cell. In certain embodiments, the first isolated nucleic acid sequence encodes a first gRNA, the gRNA being complementary to a target sequence in the integrated retroviral DNA and a second gRNA that is complementary to a target sequence in a gene encoding for at least one receptor used by a retrovirus for attachment and/or infection of a cell. In certain embodiments, the at least one receptor comprises CXCR5, variants or combinations thereof.

In certain embodiments, the first and second isolated nucleic acid sequences encode combinations of gRNAs having complementarity to one or more target sequences, the target sequences comprising retroviral DNA sequences, and sequences in one or more genes encoding for at least one receptor used by a retrovirus for attachment and/or infection of a cell.

In certain embodiments, the target sequence comprises one or more nucleic acid sequences in coding and non-coding nucleic acid sequences of the retrovirus genome.

In certain embodiments, the target sequences comprise one or more nucleic acid sequences in HIV comprising: long terminal repeat (LTR) nucleic acid sequences, nucleic acid sequences encoding structural proteins, non-structural proteins or combinations thereof.

In certain embodiments, the sequences encoding structural proteins comprise nucleic acid sequences encoding: Gag, Gag-Pol precursor, Pro (protease), Reverse Transcriptase (RT), integrase (In), Env or combinations thereof.

In certain embodiments, the sequences encoding non-structural proteins comprise nucleic acid sequences encoding: regulatory proteins, accessory proteins or combinations thereof.

In certain embodiments, the regulatory proteins comprise: Tat, Rev or combinations thereof.

In certain embodiments, the accessory proteins comprise Nef, Vpr, Vpu, Vif or combinations thereof.

In certain embodiments, the gRNA target sequences comprise one or more target sequences in an LTR region of an HIV proviral DNA and one or more target sequences in a structural gene of the HIV proviral DNA; or, one or more targets in a second gene; or, one or more targets in a first gene and one or more targets in a second gene; or, one or more targets in a first gene and one or more targets in a second gene and one or more targets in a third gene; or, one or more targets in a second gene and one or more targets in a third gene or fourth gene; or, any combinations thereof.

Guide RNA Sequences: The compositions and methods of the present disclosure may include a sequence encoding a guide RNA that is complementary to a target sequence in HIV. The genetic variability of HIV is reflected in the multiple groups and subtypes that have been described. A collection of HIV sequences is compiled in the Los Alamos HIV databases and compendiums (hiv.lanl.gov). The methods and compositions of the disclosure can be applied to HIV from any of those various groups, subtypes, and circulating recombinant forms. These include for example, the HIV-1 major group (often referred to as Group M) and the minor groups, Groups N, O, and P, as well as but not limited to, any of the following subtypes, A, B, C, D, F, G, H, J and K. or group (for example, but not limited to any of the following Groups, N, O and P) of HIV.

A gRNA includes a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). In the present disclosure, the crRNA and tracrRNA can be expressed separately or engineered into an artificial fusion gRNA via a synthetic stem loop (AGAAAU) to mimic the natural crRNA/tracrRNA duplex. Such gRNA can be synthesized or in vitro transcribed for direct RNA transfection or expressed from U6 or H1-promoted RNA expression vector.

In the compositions of the present disclosure, each gRNA includes a sequence that is complementary to a target sequence in a retrovirus. The exemplary target retrovirus is HIV, but the compositions of the present disclosure are also useful for targeting other retroviruses, such as HIV-2 and simian immunodeficiency virus (SIV)-1. The guide RNA can be a sequence complimentary to a coding or a non-coding sequence (i.e., a target sequence). For example, the guide RNA can be a sequence that is complementary to a HIV long terminal repeat (LTR) region.

Some of the exemplary gRNAs of the present disclosure are complimentary to target sequences in the long terminal repeat (LTR) regions of HIV. The LTRs are subdivided into U3, R and U5 regions. LTRs contain all of the required signals for gene expression and are involved in the integration of a provirus into the genome of a host cell. For example, the basal or core promoter, a core enhancer and a modulatory region is found within U3 while the transactivation response element is found within R. In HIV-1, the U5 region includes several sub-regions, for example, TAR or trans-acting responsive element, which is involved in transcriptional activation; Poly A, which is involved in dimerization and genome packaging; PBS or primer binding site; Psi or the packaging signal; DIS or dimer initiation site. Accordingly, in some embodiments, gRNA targets comprise one or more target sequences in an LTR region of an HIV proviral DNA and one or more targets in a structural gene of the HIV proviral DNA; or, one or more targets in a second gene; or, one or more targets in a first gene and one or more targets in a second gene; or, one or more targets in a first gene and one or more targets in a second gene and one or more targets in a third gene; or, one or more targets in a second gene and one or more targets in a third gene or fourth gene; or, any combinations thereof. Furthermore, gRNA targets directed to one or more sequences encoding a receptor for viral entry, e.g. CCR5.

Some of the exemplary gRNAs of the present disclosure target sequences in the coding and non-coding protein coding genome of HIV. gRNAs complementary to LTR target sequences include LTR 1, LTR 2, LTR 3, LTR A, LTR B, LTR B′, LTR C, LTR D, LTR E, LTR F, LTR G, LTR H, LTR I, LTR J, LTR K, LTR L, LTR M, LTR N, LTR O, LTR P, LTR Q, LTR R, LTR S, AND LTR T. gRNAs complementary to Gag target sequences include Gag A, Gag B, Gag C, and Gag D. gRNAs complementary to pol target sequences include Pol A and Pol B. Accordingly, the compositions of the present disclosure include these exemplary gRNAs, but are not limited to them, and can include gRNAs complimentary to any suitable target site in the protein coding genes of HIV, including but not limited to those encoding the envelope protein env, the structural protein tat, and the accessory proteins vif, willef (negative factor) vpu (Virus protein U) and tev.

Guide RNA sequences according to the present disclosure can be sense or anti-sense sequences. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR 3) and Neiseria meningitidis requires 5′-NNNNGATT). The specific sequence of the guide RNA may vary, but, regardless of the sequence, useful guide RNA sequences will be those that minimize off-target effects while achieving high efficiency and complete ablation of the genomically integrated HIV-1 provirus. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. Useful selection methods identify regions having extremely low homology between the foreign viral genome and host cellular genome including endogenous retroviral DNA, include bioinformatic screening using 12-bp+NGG target-selection criteria to exclude off-target human transcriptome or (even rarely) untranslated-genomic sites; avoiding transcription factor binding sites within the HIV-1 LTR promoter (potentially conserved in the host genome); and WGS, Sanger sequencing and SURVEYOR assay, to identify and exclude potential off-target effects.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs.

Combinations of gRNAs are especially effective when expressed in multiplex fashion, that is, simultaneously in the same cell. In many cases, the combinations produce excision of the HIV provirus extending between the target sites. The excisions are attributable to deletions of sequences between the cleavages induced by the endonuclease at each of the multiple target sites. These combinations pairs of gRNAs, with one member being complementary to a target site in an LTR of the retrovirus, and the other member being complementary to a gRNA complementary to a target site in a structural gene of the retrovirus. Exemplary effective combinations include Gag D combined with one of LTR 1, LTR 2, LTR 3, LTR A, LTR B, LTR C, LTR D, LTR E, LTR F, LTR G; LTR H, LTR I, LTR J, LTR K, LTR L, LTR M; LTR N, LTR O, LTR P, LTR Q, LTR R, LTR S, or LTR T. Exemplary effective combinations also include LTR 3 combined with one of LTR-1, Gag A; Gag B; Gag C, Gag D, Pol A, or Pol B.

(CR5: Macrophage (M-tropic) strains of HIV-1 use the β-chemokine receptor CCR5 for binding and are able to infect macrophages, dendritic cells, and CD4 T-cells. Almost all HIV-1 isolates are successfully transmitted using the CCR5 co-receptor. M-tropic HIV replicates in peripheral blood lymphocytes and does not form syncytia. Syncytia are ‘giant cells’, multicellular clumps that have been formed by fusing with other cells. Non-syncytia-inducing (NSI) strains of virus are considered less virulent than those that do form syncytia.

Some people have a 32-base pair deletion (delta 32) in the gene that encodes the CCR5 receptor. If they receive this deletion from both parents, they are said to be homozygous for CCR5-delta32. This deletion is highly protective because the receptor is faulty and HIV cannot use it to enter the cell.

There have been a few cases in which someone homozygous for the deletion was infected with dual-tropic HIV and suffered rapid depletion of CD4 T-cells. This is the exception. Ordinarily, it is a great advantage to have this deletion. If someone inherits the deletion from just one parent, they are said to be heterozygous for CCR5 and this can slow HIV progression. The prevalence of 32-base pair deletion is estimated to be as high as 10 to 15% in Caucasians, but only around 2% in African Americans and almost non-existent in native Africans and East Asians.

Other mutations in CCR5 that effect disease progression have also been identified, including some that might play a protective role in HIV acquisition or progression in non-Caucasian people. Slower disease progression is also associated with high levels of the CCR5 59353-C polymorphism in the promoter DNA that controls the amount of CCR5 that cells produce.

Variations also occur in the amount of chemokines in people's blood. Chemokines compete with HIV for chemokine receptors, preventing HIV from using the receptors and reducing the susceptibility of cells to infection. Unusually high levels of the CCR5-using chemokines RANTES, MIP-1 alpha, and MIP-1 beta are seen in long-term non-progressors, as well as in exposed seronegative individuals (people with repeated exposure to the virus through unprotected sex who do not become infected).

In certain embodiments, a gRNA is complementary to one or more target sequences of human CCR5 gene (NCBI Reference Sequence NG_012637.1).

CXCR4 chemokine receptors: Depending on their structure, chemokines are classified as C-C chemokines (containing a cysteine-cysteine motif) or C-X-C chemokines (containing a cysteine-X-cysteine motif). Receptors that bind such chemokines thus are classified as members of the CCR family or CXCR family, respectively. One member of the CXCR family is CXCR4, a seven transmembrane G-protein coupled receptor that is predominantly expressed on lymphocytes and that activates chemotaxis. CXCR4 binds the chemokine CXCL12 (SDF-1).

CXCR4 plays a role in embryogenesis, homeostasis and inflammation. Studies with mice engineered to be deficient in CXCR4 or SDF-1 implicate the CXCR4/SDF-1 pathway in organ vascularization, as well as in the immune and hematopoietic systems (Tachibana, K. et al. 9. (1998) Nature 393:591-594). Moreover, CXCR4 has been shown to function as a coreceptor for T lymphotrophic HIV-1 isolates (Feng, Y. et al. (1996) Science 272:872-877). CXCR4 also has been shown to be expressed on a wide variety of cancer cell types. Additionally, the CXCR4/SDF-1 pathway has been shown to be involved in stimulating the metastatic process in many different neoplasms (Murphy, P. M. (2001) N. Engl. J. Med. 345:833-835). For example, CXCR4 and SDF-1 have been shown to mediate organ-specific metastasis by creating a chemotactic gradient between the primary tumor site and the metastatic site (Muller, A. et al. (2001) Nature 410:50-56; Murakami, T. et al. (2002) Cancer Res. 62:7328-7334; Hanahan, D. et al. (2003) Cancer Res. 63:3005-3008).

These are only meant as examples and are not to be construed as limiting the disclosure in any way. When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the gRNA sequences or in a separate vector.

The gRNA sequences according to the present disclosure can be complementary to either the sense or anti-sense strands of the target sequences. They can include additional 5′ and/or 3′ sequences that may or may not be complementary to a target sequence. They can have less than 100% complementarity to a target sequence, for example 75% complementarity. The gRNA sequences can be employed as a combination of one or more different sequences, e.g., a multiplex configuration. Multiplex configurations can include combinations of two, three, four, five, six, seven, eight, nine, ten, or more different guide RNAs.

Modified or Mutated Nucleic Acid Sequences: In some embodiments, any of the nucleic acid sequences may be modified or derived from a native nucleic acid sequence, for example, by introduction of mutations, deletions, substitutions, modification of nucleobases, backbones and the like. The nucleic acid sequences include the vectors, gene-editing agents, gRNAs, etc. Examples of some modified nucleic acid sequences envisioned for this disclosure include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH, —N(CH₃)—O—CH₂[known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N⁶ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° ° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the disclosure involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO) J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington DC). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methy 1-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethy 1-2′-O-methyluridine; 5-methoxycarbonylmethy 1-2′-thiouridine; 5-carbamoylmethyluridine; 5-carbamoylmethy 1-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethy 1-2-thiouridine; 5-methylaminomethy 1-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶Nmethyladenosine; N⁶,N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopenteny 1)-adenosine; N⁶-glycinylcarbamoyl)adenosine; N⁶ threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1,2′-O-dimethyl inosine; 2′-O-methyl guanosine; 1-methyl guanosine; N²-methyl guanosine; N²,N²-dimethyl guanosine; N², 2′-O-dimethyl guanosine; N²,N², 2′-O-trimethyl guanosine; 2′-O-ribosyl guanosine (phosphate); 7-methyl guanosine; N², 7-dimethyl guanosine; N²,N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

The isolated nucleic acid molecules of the present disclosure can be produced by standard techniques. For example, polymerase chain reaction (PCR) techniques can be used to obtain an isolated nucleic acid containing a nucleotide sequence described herein. Various PCR methods are described in, for example, POR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

LASER ART: Long-acting slow effective release ART (LASER ART or LA-ART) enable improved pharmacokinetic profiles and reservoir targeting. These antiretrovirals (ARVs) overcome limitations of current drugs associated with in vivo delivery and tissue penetrance. The gene editing agent also had improved delivery and improved the therapeutic index of the drugs.

Dolutegravir, lamivudine, abacavir and rilpivirine (DTG, 3TC, ABC and RPV respectively were transformed into long-acting drugs. Drug solubility, dissolution, metabolism, protein-binding, and excretion rates for each of the antiretroviral drugs were optimized and each were shown to influence the drug's half-life and biodistribution profiles. These studies provided the means to transform standard daily or twice-daily antiretroviral drugs into hydrophobic drug crystals to extend the drug's half-life and alter its solubility and metabolic patterns. The drugs were found to possess significant antiretroviral efficacy and high tolerability for conversion into a long-acting compound. Reversible chemical modification and polymer coating techniques were developed to convert each into a long-acting nanoformulation. Change of the antiretroviral drug (ARV) structure was made through reversible myristoylation of the native compound creating a water insoluble prodrug with commensurate crystal formation. When the drug crystals were packaged into a nanoparticle, they were rapidly taken up by human monocyte-derived macrophages (MDM), slowly released from the cells, and retained for a prolonged period inside the macrophage. These chemical and biological outcomes improved drug bioavailability and increased in vitro antiretroviral activity up to 100-fold. Pharmacokinetic and pharmacodynamic profiles were improved up to 10-fold over a native drug formulation, exhibiting broad tissue distribution and increased potency. The studies herein provide evidence that ARV conversion into a long-acting slow release formulation is readily achieved. As such, the drug-encased nanoparticles were employed as a “first-step” measure to facilitate drug penetrance into viral reservoirs to facilitate the actions of the excision Cas9 system.

Accordingly, in certain embodiments, the anti-retroviral agents are formulated into long-acting nanoformulated agents or compounds.

The method represents a solution to the problem of integrated provirus, a solution which is essential to the treatment and prevention of AIDS and other retroviral diseases. During the acute phase of HIV infection, the HIV viral particles are attracted to and enter cells expressing the appropriate CD4 receptor molecules. Once the virus has entered the host cell, the HIV encoded reverse transcriptase generates a proviral DNA copy of the HIV RNA and the proviral DNA becomes integrated into the host cell genomic DNA. It is this HIV provirus that is replicated by the host cell, resulting in the release of new HIV virions which can then infect other cells.

The primary HIV infection subsides within a few weeks to a few months, and is typically followed by a long clinical “latent” period which may last for up to 10 years. During this latent period, there can be no clinical symptoms or detectable viral replication in peripheral blood mononuclear cells and little or no culturable virus in peripheral blood. However, the HIV virus continues to reproduce at very low levels. In subjects who have treated with anti-retroviral therapies, this latent period may extend for several decades or more. Anti-retroviral therapy does not suppress low levels of viral genome expression, nor does it efficiently target latently infected cells such as resting memory T cells, brain macrophages, microglia, astrocytes and gut associated lymphoid cells. Because the compositions of the present disclosure can inactivate or excise HIV-provirus, and can prevent the infection of cells by preventing expression or function the virus receptor, the methods of treatment employing the compositions constitute a new avenue of attack against HIV-1 infection

The compositions of the present disclosure, when stably expressed in potential host cells, reduce or prevent new infection by HIV. Accordingly, the present disclosure also provides a method of treatment to reduce the risk of HIV infection in a mammalian subject at risk for infection. The method includes the steps of determining that a mammalian subject is at risk of HIV infection, administering an effective amount of the previously described pharmaceutical composition, and reducing the risk of HIV infection in the mammalian subject. Preferably, the pharmaceutical composition includes a vector that provides stable and/or inducible expression of at least one of the previously enumerated.

Pharmaceutical compositions according to the present disclosure can be prepared in a variety of ways known to one of ordinary skill in the art. For example, the nucleic acids and vectors described above can be formulated in compositions for application to cells in tissue culture or for administration to a patient or subject. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This disclosure also includes pharmaceutical compositions which contain, as the active ingredient, nucleic acids and vectors described herein, in combination with one or more pharmaceutically acceptable carriers. The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the disclosure, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The nucleic acid sequences of the disclosure can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are common latently infected reservoirs of HIV infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding the an isolated nucleic acid sequence comprising a sequence encoding a CRISPR-associated endonuclease and a guide RNA is operatively linked to a promoter or enhancer-promoter combination. Promoters and enhancers are described above.

In some embodiments, the compositions of the disclosure can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI.

The nucleic acids and vectors may also be applied to a surface of a device (e.g., a catheter) or contained within a pump, patch, or other drug delivery device. The nucleic acids and vectors of the disclosure can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions can be formulated as a nanoparticle encapsulating a nucleic acid encoding Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof, and at least one gRNA sequence complementary to a target HIV and/or to a receptor target sequence, such as CCR5; or it can include a vector encoding these components. Alternatively, the compositions can be formulated as a nanoparticle encapsulating the CRISPR-associated endonuclease the polypeptides encoded by one or more of the nucleic acid compositions of the present disclosure.

In methods of treatment of HIV-1 infection, a subject can be identified using standard clinical tests, for example, immunoassays to detect the presence of HIV antibodies or the HIV polypeptide p24 in the subject's serum, or through HIV nucleic acid amplification assays. An amount of such a composition provided to the subject that results in a complete resolution of the symptoms of the infection, a decrease in the severity of the symptoms of the infection, or a slowing of the infection's progression is considered a therapeutically effective amount. The present methods may also include a monitoring step to help optimize dosing and scheduling as well as predict outcome. In some methods of the present disclosure, one can first determine whether a patient has a latent HIV infection, and then make a determination as to whether or not to treat the patient with one or more of the compositions described herein. In some embodiments, the methods can further include the step of determining the nucleic acid sequence of the particular HIV harbored by the patient and then designing the guide RNA to be complementary to those particular sequences. For example, one can determine the nucleic acid sequence of a subject's LTR U3, R or U5 region, or pol, gag, or env genes, region and then design or select one or more gRNAs to be precisely complementary to the patient's sequences. The novel gRNAs provided by the present disclosure greatly enhance the chances of formulating an effective treatment. The gRNAs targeted to nucleic acid sequences encoding a receptor used by a virus to infect a cell would prevent further infection.

In methods of reducing the risk of HIV infection, a subject at risk for having an HIV infection can be, for example, any sexually active individual engaging in unprotected sex, i.e., engaging in sexual activity without the use of a condom; a sexually active individual having another sexually transmitted infection; an intravenous drug user; or an uncircumcised man. A subject at risk for having an HIV infection can be, for example, an individual whose occupation may bring him or her into contact with HIV-infected populations, e.g., healthcare workers or first responders. A subject at risk for having an HIV infection can be, for example, an inmate in a correctional setting or a sex worker, that is, an individual who uses sexual activity for income employment or nonmonetary items such as food, drugs, or shelter.

### Combination Therapies

Accordingly, the disclosure features compositions which include therapeutically effective amounts of at least one antiretroviral agent administered sequentially or alternately or in conjunction with a composition for inactivating a proviral DNA integrated into a host cell. This composition comprises an isolated nucleic acid sequence encoding a CRISPR-associated endonuclease and one or more isolated nucleic acid sequences encoding one or more gRNAs complementary to a target sequence in HIV or another retrovirus.

In one embodiment, the antiretroviral agent comprises viral entry inhibitors, reverse transcriptase inhibitors, protease inhibitors, and immune-based therapeutic agents.

For example, when used to treat or prevent HIV infection, the antiretroviral agent or its prodrug or pharmaceutically acceptable salt can be administered in combination or alternation with another anti-HIV agent and/or a gene-editing agent embodied herein. In general, in combination therapy, effective dosages of two or more agents are administered together, whereas during alternation therapy, an effective dosage of each agent is administered serially. The dosage will depend on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

In certain embodiments, the gene-editing compositions embodied herein are administered to a patient in combination with one or more other anti-viral agents or therapeutics. Examples include any molecules that are used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

In certain embodiments, the gene-editing compositions embodied herein are administered with one or more compositions comprising a therapeutically effective amount of a non-nucleoside reverse transcriptase inhibitor (NNRTI) and/or a nucleoside reverse transcriptase inhibitor (NRTI), analogs, variants or combinations thereof. In certain embodiments, an NNRTI comprises: etravirine, efavirenz, nevirapine, rilpivirine, delavirdine, or nevirapine. In embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In certain embodiments, a composition comprises a therapeutically effective amount of at least one NNRTI or a combination of NNRTI's, analogs, variants or combinations thereof. In certain embodiments, the NNRTI is rilpivirine. In certain embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In certain embodiments, the composition comprises a therapeutically effective amount of at least one or a combination of NRTI's, analogs, variants or combinations thereof.

### Kit

The present disclosure also includes a kit including an isolated nucleic acid sequence encoding a CRISPR-associated endonuclease, for example, a Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4 endonucleases, and at least one isolated nucleic acid sequence encoding a gRNA complementary to a target sequence in an HIV provirus and at least one isolated nucleic acid sequence encoding a gRNA complementary to a target sequence in a gene or nucleic acid sequence encoding a receptor that is used by a virus to infect a cell. Alternatively, at least one of the isolated nucleic acid sequences can be encoded in a vector, such as an expression vector. Possible uses of the kit include the treatment or prophylaxis of HIV infection. Preferably, the kit includes instructions for use, syringes, delivery devices, buffers sterile containers and diluents, or other reagents for required for treatment or prophylaxis. The kit can also include a suitable stabilizer, a carrier molecule, a flavoring, or the like, as appropriate for the intended use.

### EXAMPLES

### Example 1: CRISPR/Cas9 Targeting of the CCR5 Gene

CRISPR/Cas9 mediated virus sterilization strategy was combined with loss-of-function mutation of the human CCR5 gene, a critical HIV-1 entry co-receptor. A similar approach was taken in the HIV-1 eradication study: a pair of gRNAs (called here CCR5-A and CCR5-B) to target 5′ and 3′ regions of, in this case, the human CCR5 gene located on chromosome 3. Simultaneous CRISPR/Cas9 mediated cleavage at the targets sites A and B led to massive, 768 bp long deletion of the CCR5 gene (1768) and loss of CCR5 expression in transfected HEK 293T cells. The vector's efficacy and safety were tested in transfection followed by puromycin selection of single-cell clones in the TZM-bl reporter cell line. ri768 mutation was detected in a total of six single cells clones. Most importantly, all clones carrying CCR5 ri768 mutation were resistant to infections by R5-tropic HIV-1BAL-GFP and HIV-1JR-FL. To rule out any unwarranted off-target effects, the single-cell clones carrying confirmed by sequencing, triple on target CRISPR/Cas9 mediated cleavage (CCR5-A and CCR5-B) together with two control (pX601-no gRNA) clones were screened by PCR followed by Sanger sequencing for the presence of any InDel mutations at the total 12 top predicted/possible off-target sites in the human genome. No mutations were detected. Next, the CRISPR-CCR5A+B construct was packaged into AAV6 and used sequentially in combination with the AAV9-CRISPR vector targeting HIV-1 genome (LTR and gag) using the huCD34-mice model of HIV-1 infection. 60% of animals from the dual treatment group (6/10) show undetectable virus levels in plasma and several tissues, including the brain. Combining CRISPR-based targeting of viral genome with targeting CCR5 doubled the number of cured animals in this model. Additionally, simultaneous targeting of the virus and viral entry receptor gene treatment is beneficial for both uninfected and infected cells (eradication of viral sequences and rendering cells resistant to new infections).

### Example 2: Dual CRISPR Treatment for Editing of HIV-1 and CCR5 in a Pre-Clinical Setting

A functional cure of several human immunodeficiency virus type one (HIV-1) infected persons has now been documented^{1,2}. In these cases, allogenic hematopoietic stem-cell transplant was completed from donors with either a heterozygous or homozygous mutation in the HIV-1 chemokine co-receptor type 5 (CCR5 Δ32) gene³. This facilitated treatment of acute myeloid leukemia that had developed in the infected patient. Subsequent success was attempted but stalled by a combination of pathobiological evident that included spread of infection, the numbers of viral susceptible cells, limited therapeutic access to viral reservoirs, and the inability to eliminate latent integrated proviral DNA. In these case(s) viral rebound occurred after cessation of antiretroviral therapy (ART)⁴⁻⁸. Viral elimination has not occurred by ART despite its abilities to potently suppress viral replication. The deployment of broadly neutralizing antibodies (bnAbs) while reducing HIV-1 levels and the numbers of infected cells⁹ has proven of limited value to eliminate virus based on HIV-1 resistance patterns. Moreover, HIV-1 establishes latency in long-lived CD4+ T cells and mononuclear phagocytes (MPs; monocytes, macrophages and dendritic cells). Latent infection ensued by host cell genomic viral DNA integration, remains the principal barrier to cure.

To overcome the barrier, a gene editing strategy was developed herein using CRISPR technology that eliminates the viral genome in latently infected HIV cell lines and blood cells from people with HIV (PWH). In an extension of cell line studies, as a proof-of-concept, by eliminating replication competent virus in CD34-humanized mice whose HIV-1 was controlled by long-acting, slow effective release ART, it was demonstrated that complete elimination of virus by CRISPR is possible in a fraction of HIV-1 infected animals. The goal of the current research was to. To improve the efficiency and percentage of viral clearance CRISPR technology was used to both inactivate CCR5, the orchestrator of HIV-1 target cell entry, and excise proviral DNA from the infected cells. It was reasoned that this would establish conditions of optimal viral suppression by combinatorial long-acting ART during early infection, followed by injection of dual CRISPR, one targeting the viral co-receptor CCR5, and the other the LTR Gag administered in sequential manner. The overall directive is based on the fact that viral elimination can be enhanced by abrogating a principal viral co-receptor used to block “spread of infection” then by clearance of residual integrated HIV-1 DNA by gene editing. This strategy was thus designed to enhance the efficiency of proviral DNA elimination in the infected mice¹¹⁻¹⁵.

### Results

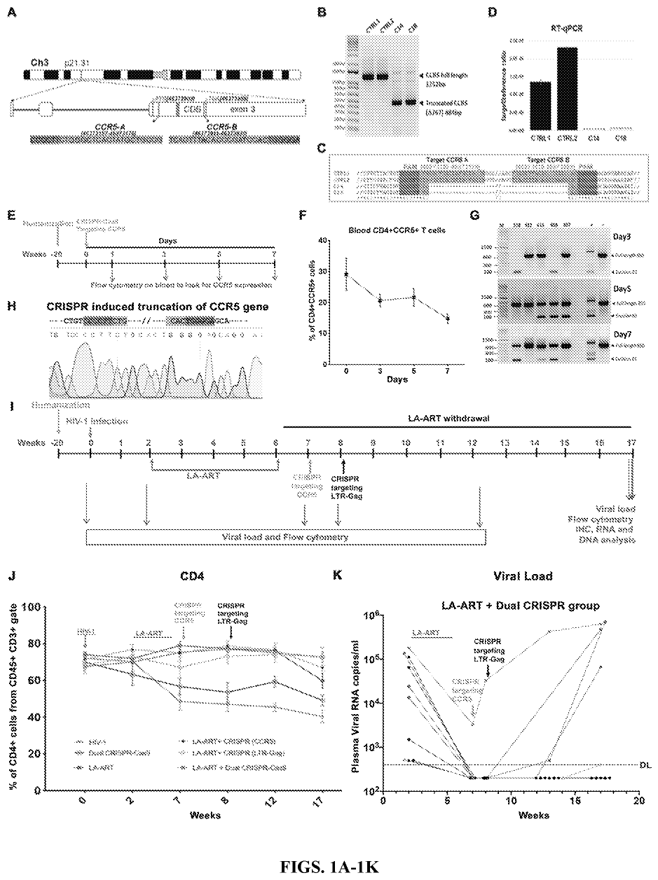

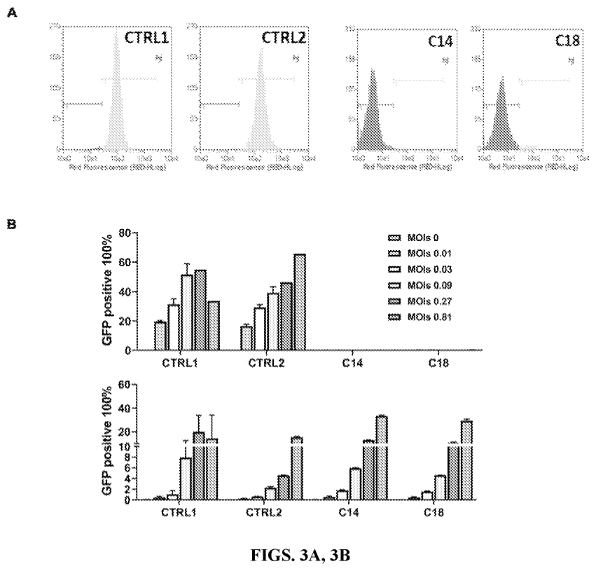

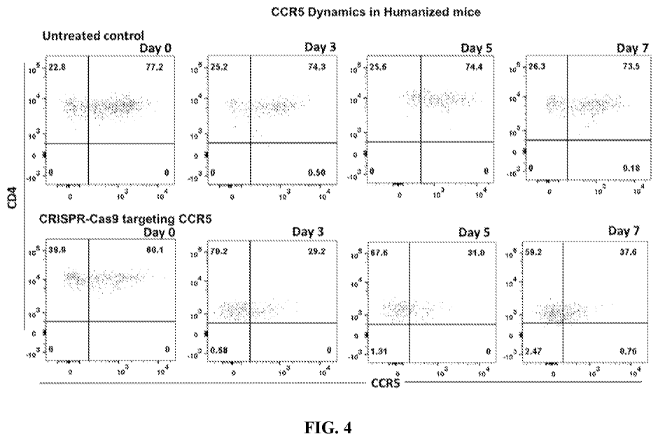

A pair of gRNAs that target specific regions within CCR5 were developed using a bioinformatic approach that avoids any off-target effects and assessed its ability to edit two sites within the CCR5 gene and excise intervening DNA sequences (nucleotides 46373019 to 46373971 in Ch3, GRCH38.p14, FIG. 1A) using TZMb1 cells. As noted, gene editing by CRISPR-Cas9 was confirmed by PCR genotyping, and the resultant truncated (484 bp) amplicons (FIG. 1B) were verified by Sanger sequencing (FIG. 1C). The lack of CCR5 expression (FIGS. 1D and 3A) protected CRISPRed cells from infection by CCR5-tropic HIV-1 (FIG. 3Bpppp). CCR5 is expressed on CD4+ T cells and cells of the myeloid lineage, serving as a principal means for HIV-1 entry into its target cells. The expression may be altered without affecting immune function¹⁶. To assess CRISPR gene editing abilities to edit CCR5, expression was examined in human immunocytes from control (uninfected) humanized mice. Mice were injected with CCR5 CRISPR-Cas9 delivered by AAV₆ at 20 weeks of age by a single intravenous (IV) tail vein dose (1012 GC units). Peripheral blood mononuclear cells were then collected at days 1, 3, 5 and 7, and examined by flow cytometry for CCR5 expression (FIGS. 1E and 4). Results demonstrated a decline in the level of CCR5 expression in CD45⁺CD3⁺CD4⁺ T cells with a peak reduction at seven days (FIG. 1F). The presence of CRISPR-induced truncated DNA was confirmed by PCR and genomic sequencing (FIGS. 1G and H). The experiments supported the notion that CRISPR-Cas9 CCR5 could affect viral spread thereby reducing the target viral DNA for HIV-1 LTR-Gag deletion¹⁰.

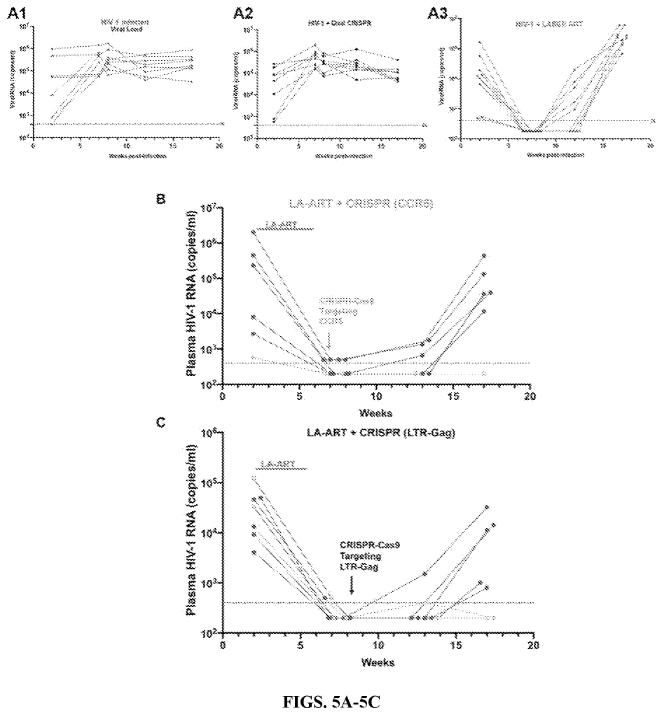

Following viral infection, a three-step treatment regimen was implemented that included ART treatment for suppression of proviral replication followed by administration of AAV₆/CRISPR-Cas9 for editing the CCR5 gene mitigating viral spread then removing targeted proviral DNA fragments by AAV₉/CRISPR-Cas9 tailored to cleave the LTR and Gag genes as illustrated in FIG. 1I. To begin, humanized mice (hu-mice) were infected with 1.5×10⁴ tissue culture infective dose₅₀ (TCID₅₀) of HIV-1_{ADA}. Plasma viral RNA were recorded at a median of 1.6×10⁵±4.4×10⁴ copies/ml (FIG. 1J). Semi-nested real-time qPCR confirmed viral infection. Two-weeks following infection, animals were divided into six groups. The first received no treatment (n=8); the second received both CCR5 and HIV-1 CRISPR-Cas9 at one-week intervals at 8 and 9 weeks following viral infection (n=8); the third received 40-45 mg/kg ART [with myristoylated nanoformulated cabotegravir (CAB)¹⁷, lamivudine (3TC)¹⁸ and abacavir (ABC)¹⁹ prodrugs and rilpivirine (RPV)¹⁰] (n=9); the fourth received an identical ART regimen followed by a single intravenous (IV) injection of AAV₉-CRISPR-Cas9 targeting LTR-Gag at week-8 (n=7); the fifth group received the ART regimen and one single IV injection of AAV₆-CRISPR-Cas9 targeting CCR5 (week 7, n=6); and the sixth received ART and dual CRISPR (weeks 7 and 8, n=10). Three uninfected mice were used as a negative control. Animals were observed for 11 weeks after ART cessation for evidence of viral rebound. Restoration of CD4⁺ T cell counts (72.6±7) was observed in all ART treated mice. CD4+ T cells were highest in group 6 as compared to group 3 animals (59.7±11.5). Group 4 (70±10) and 5 (66±4) animals showed intermediate counts. In the absence of ART, group 2 animals, CD4⁺ T cell levels were low (44.3±6) but modestly higher than those in group 1 (39%) (FIG. 1J). Viral rebound in plasma was not observed in animals in group 6 (animal numbers 345, 354, 355, 365 and 373). One animal, 392 had 400 HIV-1 RNA copies/ml that received ART and dual CRISPR-Cas9, 2 animals in the ART and CRISPR (LTR Gag), (numbers 716 and 824) and one animal in the ART and CRISPR CCR5 group 5 (792) (FIGS. 1K and 5A-5C).

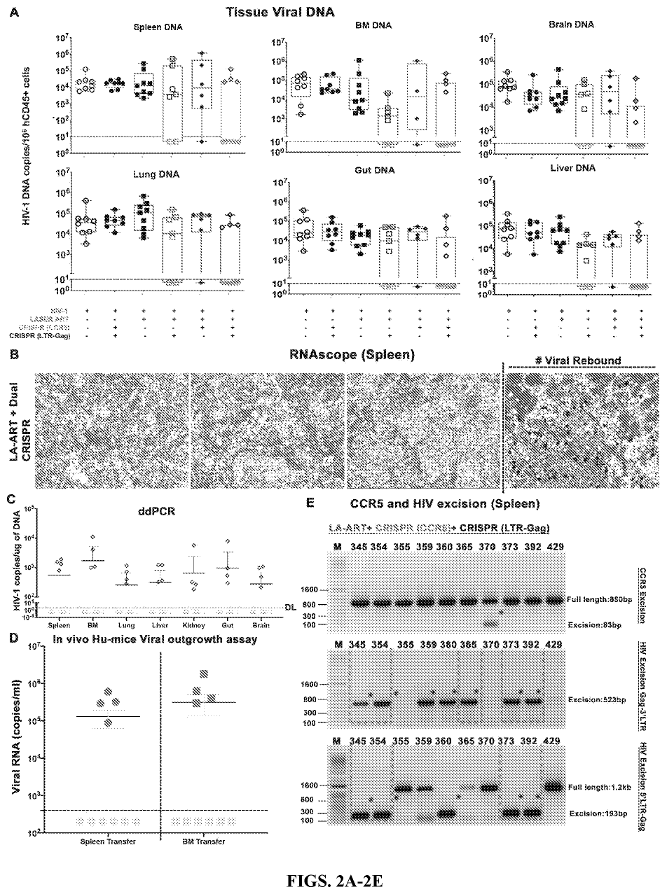

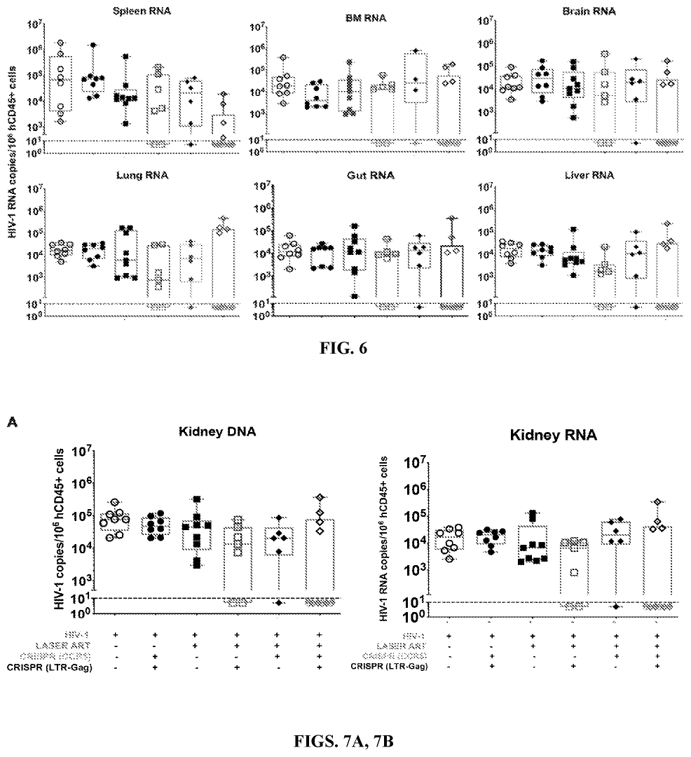

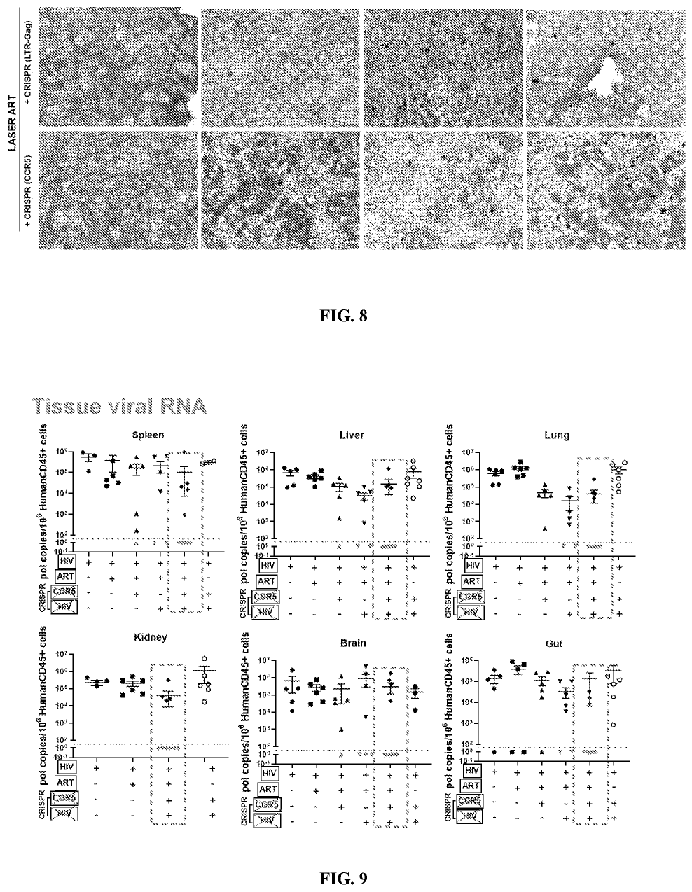

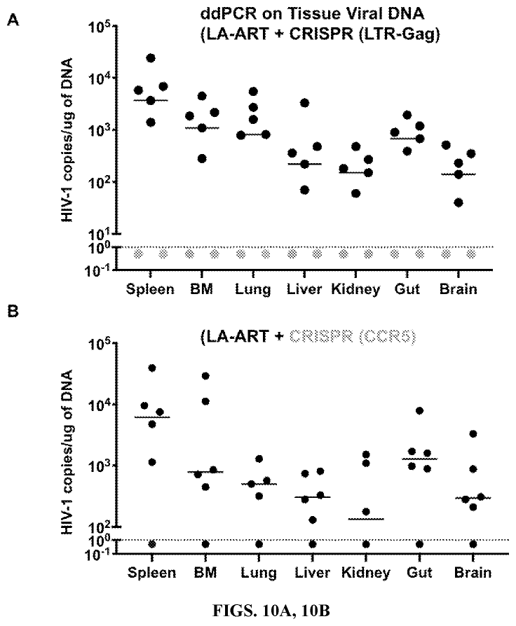

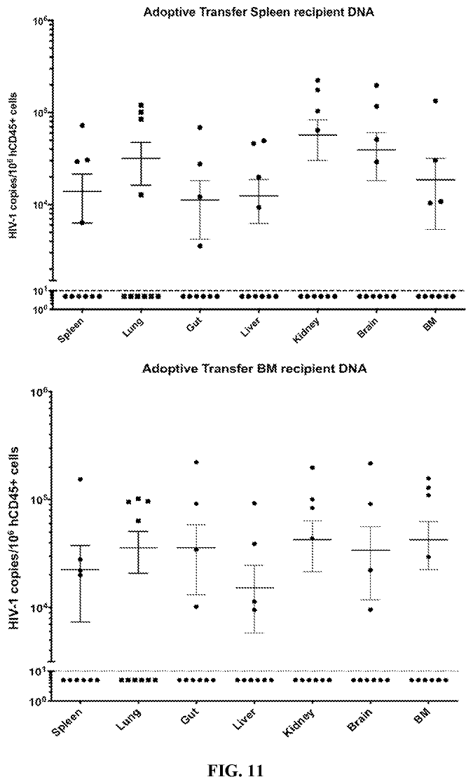

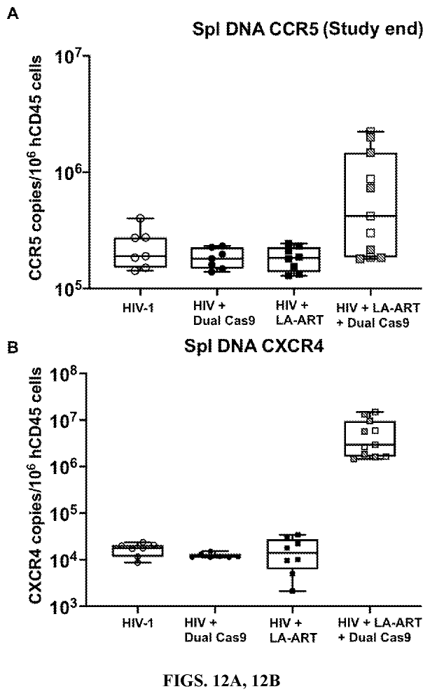

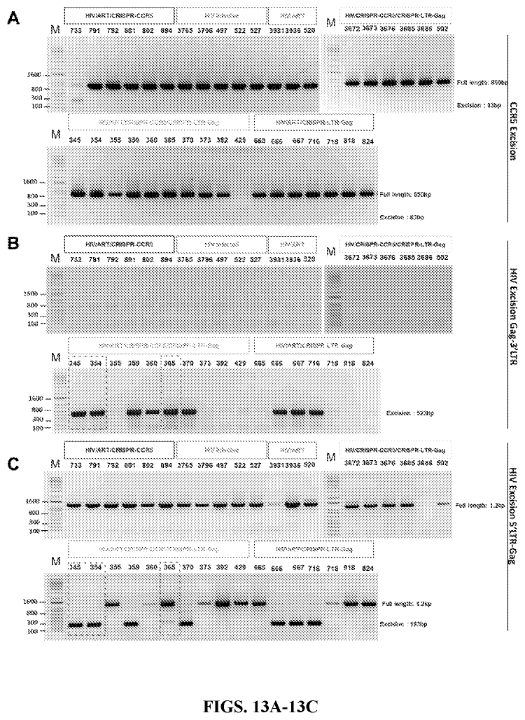

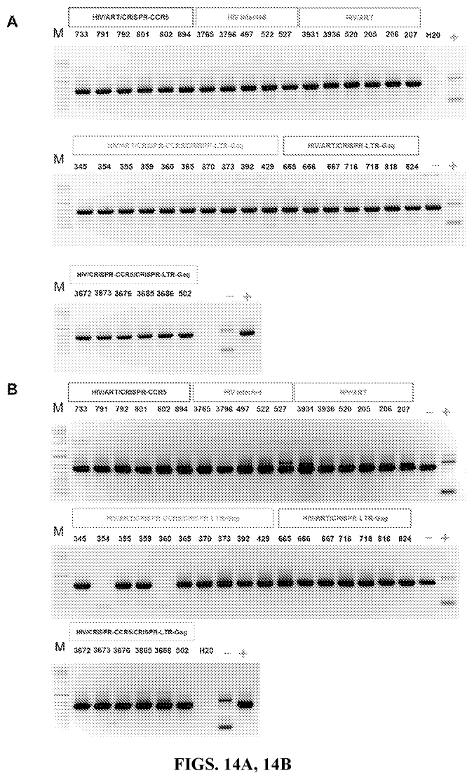

Viral DNA and RNA levels were determined in infected mouse tissues by semi-nested real-time qPCR with primers and probes designed to detect HIV-1gag^{10,20}. Combinatorial treatment (ART+CCR5 and HIV-1 CRISPR) was more effective than either ART or CRISPR-Cas9 alone in reducing viral DNA. Spleen, gut, bone marrow, lung, liver, kidney and brain of 6 mice treated with ART and dual CRISPR therapies showed no viral DNA. This is in contrast to the animal groups treated with ART and CRISPR HIV-1, and ART and CRISPR-CCR5 (groups 4 and 5, FIGS. 2A, 6, 7A and 7B). Similarly, combination of ART and dual CRISPR-Cas9 treatments (group 6) demonstrated absence of viral RNA (FIGS. 6, 7A and 7B). To affirm these test results, HIV-1 RNA was then examined by RNAscope. The assays were performed in 5 μm thick spleen sections using antisense probe designed for targeting 854-8291 HIV-1 base pairs. Under the current experimental conditions, 6 of 10 mice in dual CRISPR group and 2 out of 7 and 1 out of 6 in the single CRISPR groups (HIV-1 and CCR5, respectively) showed no evidence for viral nucleic acid and rebound (FIGS. 2B and 8). Results from the targeted qPCR for HIV-1 DNA corresponding to the pol gene (FIGS. 9, 10A and 10B) affirmed the test results. Further evidence for the absence of the HIV-1 DNA in the 6 dual CRISPR animals was provided through droplet digital PCR (ddPCR) tests. Verifying prior results, no viral DNA was detected in any animal tissues examined (FIGS. 2C and 11).

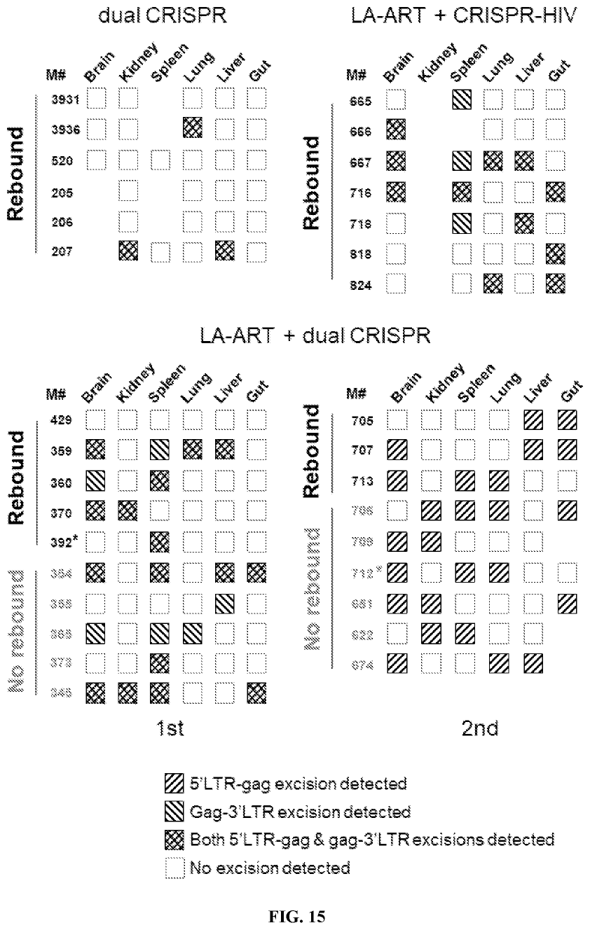

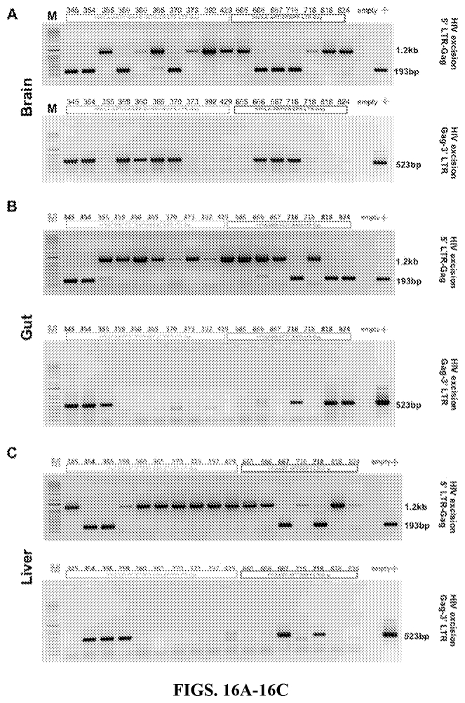

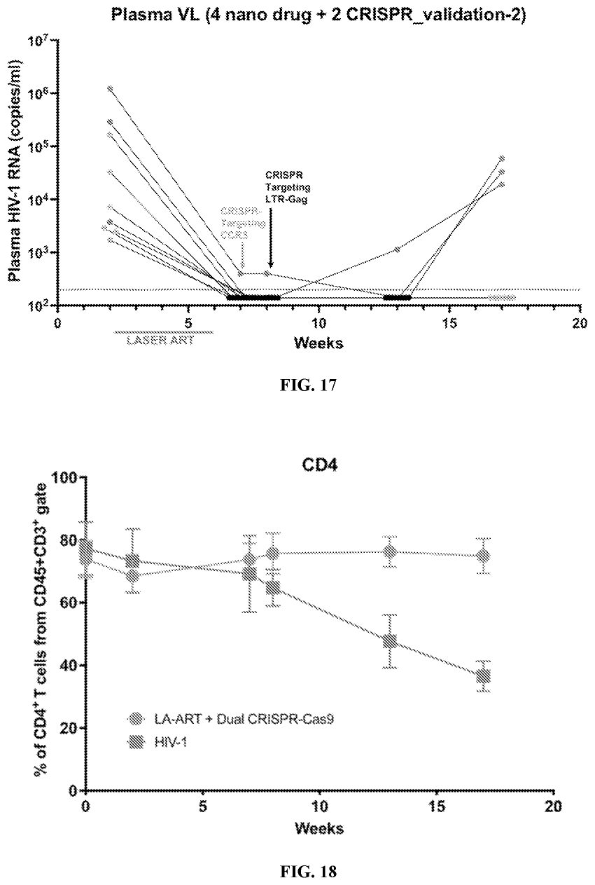

To further confirm these results, viral outgrowth assays (VOA)^{10,21} were adapted. Here adoptive transfer of bone marrow cells and splenocytes was performed from samples collected from dual and single treatment groups to humanized mice^{10,21}. No evidence for progeny virus recovery was observed in the plasma and tissues from the six animals who showed no evidence of viral DNA and RNA (FIGS. 2D, 12A and 12B). Gel electrophoretic analysis of the PCR amplified DNA fragments using specific pairs of primers designed for detection of the various cleavage events revealed robust cleavage and excision of viral DNA fragments obtained from spleen (FIG. 2E, bottom 2 panels) of the group of animals treated with LA-ART dual CRISPR-Cas9 (FIG. 2B). Excision analysis to look for CCR5 truncation didn't detect any bands (FIG. 2E upper panel), as CCR5 expression returned to baseline levels based on the repopulation of immune cells as confirmed by real-time qPCR from dual CRISPR treated spleen and lung tissues (FIGS. 12A and 12B). Removal of the predicted viral DNA fragments in other tissues including gut, liver and brain was also observed in some of the animals with dual treatments (FIGS. 14A, 14B, 15, 16A-16C and 17) and single CRISPR targeting LTR-Gag. The integrity and precision of the HIV-1 DNA excision by CRISPR-Cas9 were sequence verified (FIG. 1I). Amplification of the DNA fragments corresponding to control actin gene in the tissues was confirmed. Expression of gRNAs and Cas9 were verified. Results from sequencing of several selected sites with high scores of specificities and/or their locations in the exons ruled out any off-target effect on genome. The findings from the current study not only showed more than 58% HIV-elimination from the ART and dual CRISPR-treated group, but also reverified the HIV-1 elimination from a subset of ART and single CRISPR animals (8/23) as shown previously¹⁰.

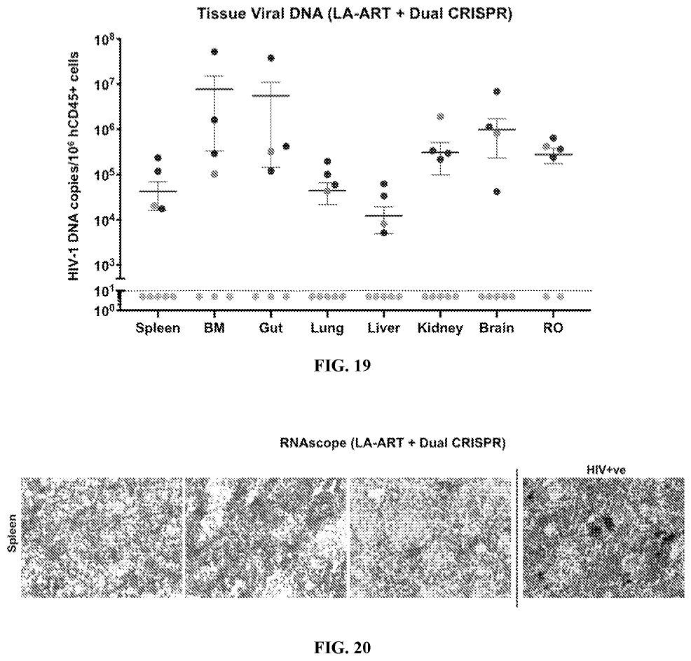

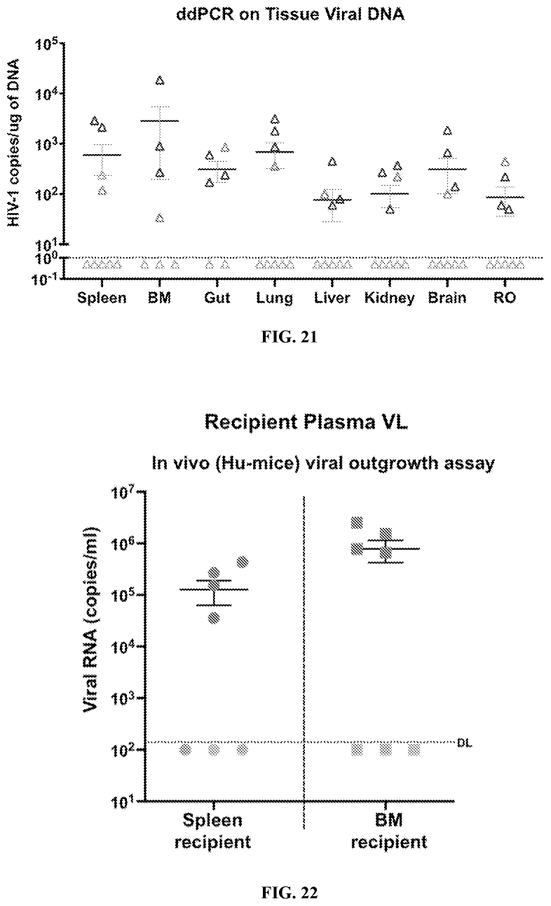

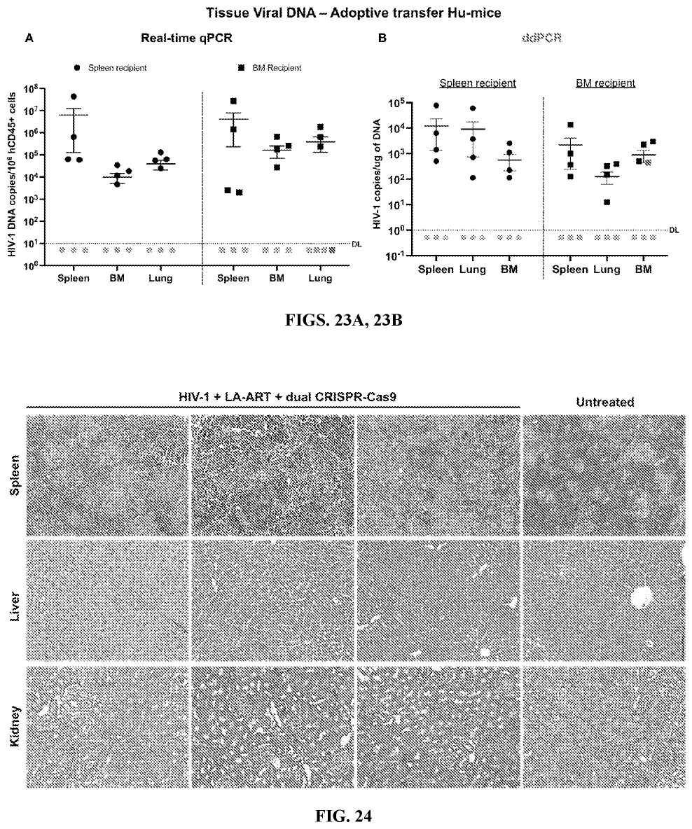

To extend and validate these observations, replicate experiments were performed with independent sets of humanized mice following a similar scheme as outlined in FIG. 1I. In this experiment, 9 humanized mice were infected with 2×10⁴ TCID₅₀ of HIV-1_{ADA} for two-weeks. After infection was confirmed, LA-ART was administered for an additional 4 weeks to minimize levels of spreading viral replication. Then the mice were treated with CRISPR-Cas9 targeting CCR5 (week 7) and LTR gag (week 8) and evaluated evidence for viral rebound 9 weeks after the last CRISPR administration (17 weeks after viral infection). Six out of nine mice were found to have undetectable plasma viremia at study end (FIG. 19). CD3⁺CD4⁺ T cells are restored in most of the animals (FIG. 20). Viral DNA and RNA amplification from tissues by semi-nested real-time qPCR (FIG. 21), RNAscope (FIG. 22) ddPCR (FIGS. 23A, 23B) identified 5/9 animals (numbers 706, 709, 622, 651 and 674) without HIV-1. Another animal, 712, had undetectable plasma viremia. However, virus was found in tissues by ddPCR. This result highlighted the need to examine tissue HIV-1 latent reservoirs before concluding that virus was eliminated. Humanized mice VOA using spleen and bone marrow tissues from the dual treated animals confirmed the absence of virus in plasma and tissue (FIG. 24). PCR analysis of different tissues from the cured animal was performed and the excised bands were verified by sequencing. No ART or CRISPR toxicities were observed by histological analyses (hematoxylin and eosin staining) of spleen, liver and kidney tissues (FIG. 24).

### CONCLUSION AND DISCUSSION

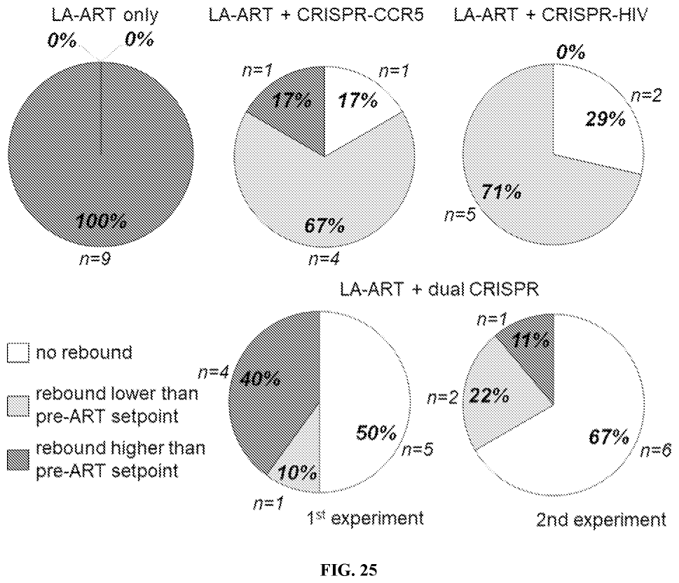

ART and CRISPR gene editing for targeting CCR5 and HIV-1 LTR Gag while controlling viral replication by antiretroviral drugs can lead to HIV-1 elimination in tissue reservoirs of infected animals. Evidence was provided for the absence of viral rebound after 11 weeks following ART cessation. These data sets were affirmed by sensitive PCR and viral rescue assays. Further improvements were demonstrated in ART and CRISPR for combination editing of viral and strategically important cellular genes such as CCR5 in in vivo hu-mice may achieve and serve as a proof-of-principle for further investigation toward clinic. Interestingly, analysis of plasma viral loads at pre-LA-ART (2WPI) and post-LA-ART withdrawal (17WPI) timepoints revealed the additional, previously unanticipated effect of combined LA-ART plus single CRISPR treatments on virus rebound. While in LA-ART-only treated animals at 17WPI, plasma viral loads were consistently higher (5×-1000×) than pre-LA-ART setpoint (2WPI), in LA-ART+CRISPR-CCR5 and LA-ART+CRISPR-HIV treatment groups majority of animals (67% and 71% respectively) showed lower than pre-LASER-ART plasma viral loads in addition to animals with lack of viral rebound (FIG. 25). The lower-than-pre-LA-ART setpoint viral rebound in CRISPR-treated animals might indicate a CRISPR-mediated reduction in the size of the reactivable reservoir due to 1) increased resistance of HIV-1 target cells to viral infection (CRISPR-CCR5) and 2) direct damage to the intact proviral genomes (CRISPR-HIV). Combining these two CRISPR-mediated antiviral strategies increased the number of animals showing a lack of rebound observed in LA-ART+dual CRISPR treatment groups. The results, taken together, provide one potential pathway for elimination of HIV-1.

### REFERENCES

1. Hutter, G., et al. Long-term control of HIV by CCR5 Delta32/Delta32 stem-cell transplantation. The New England journal of medicine 360, 692-698 (2009).
2. Gupta, R. K., et al. HIV-1 remission following CCR5Delta32/Delta32 haematopoietic stem-cell transplantation. Nature (2019).
3 Huang, Y., et al. The role of a mutant CCR5 allele in HIV-1 transmission and disease progression. Nat Med 2, 1240-1243 (1996).
4. Xu, W., et al. Advancements in Developing Strategies for Sterilizing and Functional HIV Cures. BioMed research international 2017, U.S. Pat. No. 6,096,134 (2017).
5. Saez-Cirion, A., et al. Post-treatment HIV-1 controllers with a long-term virological remission after the interruption of early initiated antiretroviral therapy ANRS VISCONTI Study. PLOS pathogens 9, e1003211 (2013).
6. Li, J. Z., et al. The size of the expressed HIV reservoir predicts timing of viral rebound after treatment interruption. AIDS (London, England) 30, 343-353 (2016).
7. Siliciano, J. D. & Siliciano, R. F. Recent developments in the effort to cure HIV infection: going beyond N=1. The Journal of clinical investigation 126, 409-414 (2016).
8 Martin, A. R. & Siliciano, R. F. Progress Toward HIV Eradication: Case Reports, Current Efforts, and the Challenges Associated with Cure. Annual review of medicine 67, 215-228 (2016).
9. Borducchi, E. N., et al. Antibody and TLR7 agonist delay viral rebound in SHIV-infected monkeys. Nature 563, 360-364 (2018).
10. Dash, P. K., et al. Sequential LASER ART and CRISPR Treatments Eliminate HIV-1 in a Subset of Infected Humanized Mice. Nat Commun 10, 2753 (2019).
11. Kaminski, R., et al. Elimination of HIV-1 Genomes from Human T-lymphoid Cells by CRISPR/Cas9 Gene Editing. Sci Rep 6, 22555 (2016).
12. Kaminski, R., et al. Negative Feedback Regulation of HIV-1 by Gene Editing Strategy. Scientific reports 6, 31527 (2016).
13. White, M. K., Hu, W. & Khalili, K. Gene Editing Approaches against Viral Infections and Strategy to Prevent Occurrence of Viral Escape. PLoS pathogens 12, e1005953 (2016).
14. Kaminski, R., et al. Excision of HIV-1 DNA by gene editing: a proof-of-concept in vivo study. Gene therapy 23, 690-695 (2016).
15. Yin, C., et al. In Vivo Excision of HIV-1 Provirus by saCas9 and Multiplex Single-Guide RNAs in Animal Models. Molecular therapy: the journal of the American Society of Gene Therapy 25, 1168-1186 (2017).
16. Heredia, A., et al. Rapamycin causes down-regulation of CCR5 and accumulation of anti-HIV beta-chemokines: an approach to suppress R5 strains of HIV-1. Proc Natl Acad Sci USA 100, 10411-10416 (2003).
17. Zhou, T., et al. Creation of a nanoformulated cabotegravir prodrug with improved antiretroviral profiles. Biomaterials 151, 53-65 (2018).
18. Guo, D., et al. Creation of a Long-Acting Nanoformulated 2′,3′-Dideoxy-3′-Thiacytidine. J Acquir Immune Defic Syndr 74, e75-e83 (2017).
19. Singh, D., et al. Development and characterization of a long-acting nanoformulated abacavir prodrug. Nanomedicine (Lond) 11, 1913-1927 (2016).
20. Arainga, M., Su, H., Poluektova, L. Y., Gorantla, S. & Gendelman, H.E. HIV-1 cellular and tissue replication patterns in infected humanized mice. Sci Rep 6, 23513 (2016).
21. Su, H., et al. Amplification of Replication Competent HIV-1 by Adoptive Transfer of Human Cells From Infected Humanized Mice. Front Cell Infect Microbiol 10, 38 (2020).

### Other Embodiments

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

What is claimed is:

1. A composition for preventing or treating a retroviral infection in vitro or in vivo, the composition comprising at least two isolated nucleic acid sequences wherein:
(i) the first isolated nucleic acid sequence encodes a first Clustered Regularly Interspaced Short Palindromic Repeat (CRISPR)-associated endonuclease and at least one guide RNA (gRNA), the gRNA being complementary to a target sequence in the integrated retroviral DNA;
(ii) the second isolated nucleic acid sequence encodes a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell in vitro or in vivo;
wherein the CRISPR-associated endonuclease, the first gRNA, and the second gRNA are capable of excising intervening sequences between the first target sequence and the second target sequence.

2. The composition of claim 1, wherein the first isolated nucleic acid sequences encode at least one gRNA, the gRNA being complementary to a target sequence in the integrated retroviral DNA and a second gRNA that is complementary to a second target sequence in the integrated retroviral DNA.

3. The composition of claim 1, wherein the second isolated nucleic acid sequence encodes a first gRNA that is complementary to a first target sequence in a gene encoding for at least one receptor used by a retrovirus for attachment and/or infection of a cell; and a second gRNA that is complementary to a second target sequence in a gene encoding for at least one receptor used by a retrovirus for attachment and/or infection of a cell.

4. The composition of claims 1-3, wherein the first isolated nucleic acid sequence encodes a first gRNA, the gRNA being complementary to a target sequence in the integrated retroviral DNA and a second gRNA that is complementary to a target sequence in a gene encoding for at least one receptor used by a retrovirus for attachment and/or infection of a cell.

5. The composition of claim 4, wherein the at least one receptor comprises CCR5, variants or combinations thereof.

6. The composition of any one of claims 1-5, further comprising administering one or more isolated nucleic acid sequence encoding one or more (CRISPR)-associated endonucleases and at least one guide RNA (gRNA) having complementarity to one or more target sequences, the target sequences comprising retroviral DNA sequences.

7. The composition of claim 6, wherein the target sequences comprise one or more nucleic acid sequences in HIV comprising: long terminal repeat (LTR) nucleic acid sequences, Gag nucleic acid sequences, nucleic acid sequences encoding structural proteins, non-structural proteins or combinations thereof.

8. The composition of claim 1, further comprising administering a therapeutically effective amount of at least one antiretroviral agent.

9. The composition of claim 8, wherein the antiretroviral agent is formulated as a long-acting slow effective release (LASER) antiretroviral agent.

10. The composition of claim 9, wherein the at least one antiretroviral agent is nanoformulated.

11. The composition of claim 10, wherein the at least one antiretroviral agent comprises: myristolyated dolutegravir, lamivudine, abacavir, rilpivirine or combinations thereof.

12. A composition comprising at least two isolated nucleic acid sequences wherein:
(iii) the first isolated nucleic acid sequence encodes a first Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell;
(iv) the second isolated nucleic acid sequence encodes a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell;
wherein the CRISPR-associated endonuclease, the first gRNA, and the second gRNA are capable of excising intervening sequences between the first target sequence and the second target sequence.

13. The composition of claim 12, wherein the at least one receptor used by a retrovirus for attachment and/or infection of a cell comprises a chemokine receptor.

14. The composition of claim 13, wherein the at least one chemokine receptor comprises CCR5, variants or combinations thereof.

15. The composition of any one of claims 1-5, further comprising administering two or more isolated nucleic acid sequence encoding one or more (CRISPR)-associated endonucleases and at least two guide RNAs (gRNAs) having complementarity to one or more target sequences, the target sequences comprising retroviral DNA sequences, wherein the CRISPR-associated endonuclease and gRNAs are capable of excising intervening sequences between the first target sequence and the second target sequence.

16. The composition of claim 15, wherein the target sequences comprise one or more nucleic acid sequences in HIV comprising: long terminal repeat (LTR) nucleic acid sequences, Gag nucleic acid sequences, nucleic acid sequences encoding structural proteins, non-structural proteins or combinations thereof.

17. The composition of claim 12, further comprising administering a therapeutically effective amount of at least one antiretroviral agent.

18. The composition of claim 17, wherein the antiretroviral agent is formulated as a long-acting slow effective release (LASER) antiretroviral agent.

19. The composition of claim 18, wherein the at least one antiretroviral agent is nanoformulated.

20. The composition of claim 19, wherein the at least one antiretroviral agent comprises: myristolyated dolutegravir, lamivudine, abacavir, rilpivirine or combinations thereof.

21. A method of inactivating or eradicating an integrated retroviral DNA and preventing infection by a retrovirus in vitro or in vivo, including the steps of exposing the cell to a composition comprising at least one isolated nucleic acid sequence encoding a gene editing complex comprising a Clustered Regularly Interspaced Short Palindromic Repeat (CRISPR)-associated endonuclease, a first guide RNA (gRNA), the first gRNA being complementary to a target sequence in the integrated retroviral DNA; a second guide RNA (gRNA), the second gRNA being complementary to a target sequence in a gene encoding for at least one receptor used by a retrovirus for attachment and/or infection of a cell in vitro or in vivo, wherein the CRISPR-associated endonuclease, the first gRNA, and the second gRNA are capable of excising intervening sequences between the first target sequence and the second target sequence.

22. The method of claim 21, further comprising administering to a subject composition comprising a therapeutically effective amount of at least one antiretroviral agent.

23. The method of claim 22, wherein the antiretroviral agent is formulated as a long-acting slow effective release (LASER) antiretroviral agent.

24. The method of claim 23, wherein the at least one antiretroviral agent is nanoformulated.

25. The method of claim 23, wherein the at least one antiretroviral agent comprises: myristolyated dolutegravir, lamivudine, abacavir, rilpivirine or combinations thereof.

26. The method of claim 22, wherein the at least one antiretroviral agent is administered to the subject prior to administering the at least one gene editing agent.

27. The method of claim 22, wherein the at least one antiretroviral agent and at least one gene-editing agent are co-administered.

28. The method of claim 22, wherein the at least one antiretroviral agent and at least one gene-editing agent are administered sequentially.

29. The method of claim 21, wherein the at least one receptor used by a retrovirus for attachment and/or infection of a cell comprises a chemokine receptor.

30. The method of claim 29, wherein the at least one chemokine receptor comprises CCR5, variants or combinations thereof.

31. The method of claim 21, further comprising administering two or more isolated nucleic acid sequence encoding one or more (CRISPR)-associated endonucleases and at least two guide RNAs (gRNAs) having complementarity to one or more target sequences, the target sequences comprising retroviral DNA sequences, wherein the CRISPR-associated endonuclease and gRNAs are capable of excising intervening sequences between the first target sequence and the second target sequence.

32. The method of claim 31, wherein the target sequences comprise one or more nucleic acid sequences in HIV comprising: long terminal repeat (LTR) nucleic acid sequences, Gag nucleic acid sequences, nucleic acid sequences encoding structural proteins, non-structural proteins or combinations thereof.

33. A method of inactivating or eradicating an integrated retroviral DNA and preventing infection by a retrovirus in vitro or in vivo, comprising administering at least two isolated nucleic acid sequences wherein:
(i) the first isolated nucleic acid sequence encodes a first Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell;
(ii) the second isolated nucleic acid sequence encodes a second Clustered Regularly Interspaced Short Palindromic Repeat (CRISPR)-associated endonuclease and at least one guide RNA (gRNA), the gRNA being complementary to a target sequence in a gene encoding for at least one receptor used by a retrovirus for attachment and/or infection of a cell;
wherein the CRISPR-associated endonuclease, the first gRNA, and the second gRNA are capable of excising intervening sequences between the first target sequence and the second target sequence.

34. The method of claim 33, wherein the at least one receptor used by a retrovirus for attachment and/or infection of a cell comprises a chemokine receptor.

35. The method of claim 34, wherein the at least one chemokine receptor comprises CCR5, variants or combinations thereof.

36. The method of any one of claims 33 to 35, further comprising administering one or more isolated nucleic acid sequences encoding one or more (CRISPR)-associated endonucleases and at least one guide RNA (gRNA) having complementarity to one or more target sequences, the target sequences comprising retroviral DNA sequences.

37. The method of claim 36, wherein the target sequences comprise one or more nucleic acid sequences in HIV comprising: long terminal repeat (LTR) nucleic acid sequences, Gag nucleic acid sequences, nucleic acid sequences encoding structural proteins, non-structural proteins or combinations thereof.

38. The method of claim 33, further comprising administering a therapeutically effective amount of at least one antiretroviral agent.

39. The method of claim 38, wherein the antiretroviral agent is formulated as a long-acting slow effective release (LASER) antiretroviral agent.

40. The method of claim 39, wherein the at least one antiretroviral agent is nanoformulated.

41. The method of claim 39, wherein the at least one antiretroviral agent comprises: myristolyated dolutegravir, lamivudine, abacavir, rilpivirine or combinations thereof.

42. The method of claim 39, wherein the at least one antiretroviral agent is administered to the subject prior to administering the at least one gene editing agent.

43. The method of claim 38, wherein the at least one antiretroviral agent and at least one gene-editing agent are co-administered.

44. The method of claim 38, wherein the at least one antiretroviral agent and at least one gene-editing agent are administered sequentially.

45. The method of claim 33, wherein the at least one receptor used by a retrovirus for attachment and/or infection of a cell comprises a chemokine receptor.

46. The method of claim 45, wherein the at least one chemokine receptor comprises CCR5, variants or combinations thereof.

47. A synthetic nucleic acid sequence comprising at least a 75% sequence identity to gRNA sequences: CCR5-A: CGGCAGCATAGTGAGCCCAG (SEQ ID NO: 1), CCR5-B: TCAGTTTACACCCGATCCAC (SEQ ID NO: 2); LTR1: GCAGAACTACACACCAGGGCC (SEQ ID NO: 3), GagD: GGATAGATGTAAAAGACACCA (SEQ ID NO: 4) and combinations thereof.

48. The synthetic nucleic acid sequence of claim 48, wherein, the gRNAs comprise CCR5-A: CGGCAGCATAGTGAGCCCAG (SEQ ID NO: 1), CCR5-B: TCAGTTTACACCCGATCCAC (SEQ ID NO: 2); LTR1: GCAGAACTACACACCAGGGCC (SEQ ID NO: 3), GagD: GGATAGATGTAAAAGACACCA (SEQ ID NO: 4) and combinations thereof.
